(19) United States
(12) Patent Application Publication
(10) Pub. No.: US 2025/0197493 A1
(43) Pub. Date: Jun. 19, 2025

(54) LAG-3 AGONIST ANTIBODIES

(71) Applicant: ELI LILLY AND COMPANY, Indianapolis, IN (US)

(72) Inventors: Deepa BALASUBRAMANIAM, San Diego, CA (US); Yaqiong LIN, Carlsbad, CA (US); David MUENCH, Carlsbad, CA (US); Songqing NA, San Diego, CA (US); Marta Antonina WITEK, Zionsville, IN (US); Jianghuai XU, San Diego, CA (US)

(21) Appl. No.: 18/978,898

(22) Filed: Dec. 12, 2024

Related U.S. Application Data
(60) Provisional application No. 63/609,523, filed on Dec. 13, 2023.

(51) Int. Cl. C07K 16/28; A61K 39/00; A61P 37/06

(52) U.S. Cl. CPC C07K 16/2803; A61P 37/06; A61K 2039/505; C07K 2317/75; C07K 2317/92

(57) ABSTRACT

The present disclosure relates to antibodies that bind to and agonize human LAG-3, and to methods for treating autoimmune disease, thereby promoting T-cell receptor signaling downregulation. Methods of making and using said agonistic antibodies also are disclosed, especially for use in the prevention, down-regulation and/or amelioration of autoimmune and/or immune tolerance related disorders.

### FIELD OF THE INVENTION

The present disclosure is in the field of medicine. Particularly, the present disclosure relates to novel antibodies that agonize human LAG-3, compositions comprising such antibodies, and methods of using such antibodies for the treatment of autoimmune disease.

### SEQUENCE LISTING

The present application is being filed along with a Sequence Listing in ST.26 XML format. The Sequence Listing is provided as a file titled “30922_WO.xml” created 6 Dec. 2024 and is 108 kilobytes in size. The Sequence Listing information in the ST.26 XML format is incorporated herein by reference in its entirety.

### BACKGROUND OF THE INVENTION

Immune checkpoint pathways modulate both the autoimmune response and the anti-cancer immune response (Isakov, N., J. Autoimmune Disorders 2016; 2 (2): 17). In autoimmune disease therapy, promoting, i.e., agonizing, the effect of an immune-inhibitory pathway, such that the immune response is suppressed, can be desirable. Lymphocyte activation gene-3 (LAG-3; CD223) was first described over 30 years ago as a cell surface protein selectively expressed on activated human NK and T cells (Triebel, F., et al., J. Exp. Med. 1990; 171:1393-1405 known to be a cell-surface inhibitory receptor that regulates T-cell effector functions (Hu, S et al., J. Autoimmunity 2020; 112:102504, doi.org/10.1016/j.jaut.2020.102504). LAG-3 is a checkpoint inhibitory receptor that disrupts the initiating phosphorylation event in T cell receptor signaling, to suppress T cell activation and function.

LAG-3 agonistic antibodies have been reported in WO 2017/037203 and WO 2020/221928, both of which disclose the LAG-3 agonist antibody, IMP761, which specifically binds to a discontinuous epitope within the extracellular Ig superfamily domain D1 of human LAG-3, wherein the epitope lies outside a 30 amino acid extra-loop sequence of domain D1 of the LAG-3 protein. More recently, WO2024189628 teaches inter alia antibodies which are directed to glycosylation sites on specific domains of LAG-3 for use for the prevention, amelioration or treatment of influenza virus infection. Despite LAG-3 being a target of high interest and decades of intense research, including many antibody generating campaigns, there are still no marketed LAG-3 agonistic antibodies, nor are there any LAG-3 agonistic antibodies in late-stage clinical trials, for use in treating autoimmune disorders. For instance, despite evidence of target cell depletion in blood, GSK2831781, a humanized IgG1 monoclonal antibody (mAb) that is specific to the LAG-protein and specifically engineered to induce targeted depletion of LAG-3 positive T cells failed to reduce inflammation in the colonic mucosa, suggesting no pharmacological effect in patients with moderate to severe active ulcerative colitis (UC). Therefore, the study (i.e., NCT03893565) was terminated early and GlaxoSmithKline subsequently terminated its development of GSK2831781 as of May 30, 2024 (D'Haens, G., et al., Aliment Pharmacol Ther. 2023; 58 (3): 283-296. doi: 10.1111/apt.17557. Epub 2023 Jun. 16.

Accordingly, there remains a need for LAG-3 agonist antibodies for treating autoimmune disease that bind to human LAG-3 with desirable association and dissociation rates for optimal agonist activity, agonize the LAG-3 signaling pathway, have reduced immunogenicity risk, do not significantly deplete T-cells, decrease T cell proliferation by promoting T-cell receptor signaling downregulation (e.g., potent suppression of CD4 and CD8 activity) instead of by depletion of T-cells, agonize human LAG-3 in an immunologically relevant context to achieve in vivo efficacy, bind potently to Fc gamma receptors, display sufficient potency as a molecule for the treatment and/or prevention of autoimmune disorders, and/or offer an effective alternative for drug switching when, during the treatment of an autoimmune disorder with another human LAG-3 agonist antibody, therapy is suspended because of at least one adverse event and/or inefficacy (particularly, anti-drug antibody (ADA) mediated reduction in efficacy).

### SUMMARY OF THE INVENTION

The present disclosure provides novel anti-human LAG-3 antibodies. The present disclosure provides an advance in the art by providing compositions and methods useful in the prevention, downregulation or amelioration of autoimmune and/or immune tolerance related disorders through immune checkpoint stimulation using an engineered human LAG-3 agonist antibody. The anti-human LAG-3 agonist antibodies of the present disclosure may be capable of improving or restoring immune homeostasis, preferably, through inhibition of the T-cell mediated arm of the immune response, selective immunosuppression of activated T cells, and, thereby directly addressing the underlying disease pathology, with reduced immunogenicity risk as compared to known LAG-3 agonist antibodies. The use of such antibodies clinically may lead to long-term durability or remission of the disease(s) being treated.

The present disclosure provides an anti-human LAG-3 antibody that binds Domain 2 of human LAG-3 (SEQ ID NO: 61), wherein the antibody binds an epitope comprising one or more amino acid residues within SPHHHLAESF (SEQ ID NO: 64) of human LAG-3 Domain 2 (SEQ ID NO: 61).

The present disclosure also provides an anti-human LAG-3 antibody that binds to human LAG-3, wherein the antibody comprises a human LAG-3 binding domain comprising a heavy chain variable region (VH) and a light chain variable region (VL), wherein the VH comprises heavy chain complementarity determining regions (HCDR):
HCDR1 comprising SEQ ID NO: 65, HCDR2 comprising SEQ ID NO: 2, and HCDR3 comprising SEQ ID NO: 48; and
the VL comprises light chain complementarity determining regions (LCDR): LCDR1 comprising SEQ ID NO: 66, LCDR2 comprising SEQ ID NO: 67, and LCDR3 comprising SEQ ID NO: 68.

In another embodiment, the present disclosure provides an anti-human LAG-3 antibody that binds to human LAG-3, wherein the antibody comprises an antigen binding domain comprising a VH and a VL, wherein the VH comprises HCDRs:
HCDR1 comprising SEQ ID NO: 1, HCDR2 comprising SEQ ID NO: 2, and HCDR3 comprising SEQ ID NO: 48; and wherein the VL comprises LCDRs:
LCDR1 comprising SEQ ID NO: 49, LCDR2 comprising SEQ ID NO: 5, and LCDR3 comprising SEQ ID NO: 6.

In one embodiment, the VH comprises SEQ ID NO: 50 and the VL comprises SEQ ID NO: 51.

In another embodiment, the anti-human LAG-3 antibody comprises a constant heavy (CH) region comprising SEQ ID NO: 9 and a constant light (CL) region comprising SEQ ID NO:
10.

The present disclosure provides an anti-human LAG-3 antibody that binds to human LAG-3, wherein the antibody comprises an antigen binding domain comprising a VH and a VL,
wherein the VH comprises HCDRs:
HCDR1 comprising SEQ ID NO: 1, HCDR2 comprising SEQ ID NO: 2, and HCDR3 comprising SEQ ID NO: 3; and
wherein the VL comprises LCDRs:
LCDR1 comprising SEQ ID NO: 4, LCDR2 comprising SEQ ID NO: 5, and LCDR3 comprising SEQ ID NO: 6.

In one embodiment, the VH comprises SEQ ID NO: 7 and the VL comprises SEQ ID NO: 8.

In another embodiment, the anti-human LAG-3 antibody comprises a CH region comprising SEQ ID NO: 9 and a CL region comprising SEQ ID NO: 10.

In another embodiment, the anti-human LAG-3 antibody comprises a LC comprising SEQ ID NO: 12 and a HC comprising SEQ ID NO: 11.

In another embodiment, an anti-human LAG-3 antibody of the invention is a LAG-3 agonist antibody.

In another embodiment, an anti-human LAG-3 antibody of the invention binds to human LAG-3 (e.g., SEQ ID NO: 57 or SEQ ID NO: 58), to a human LAG-3 ECD (e.g., SEQ ID NO: 59), and/or to a cynomolgus LAG3 ECD (e.g., SEQ ID NO: 60), with a K_{D} of about 1×10⁻⁷ M to about 5×10⁻¹¹ M as determined by methods known in the art and/or by methods essentially as described herein, including, but not limited to, use of a surface plasmon resonance (SPR) biosensor at 25° C. or 37° C. In another embodiment, an anti-human LAG-3 agonist antibody of the present disclosure binds to human LAG-3 (SEQ ID NO: 57 or SEQ ID NO: 58), to a human LAG-3 ECD (e.g., SEQ ID NO: 59), and/or to a cynomolgus LAG3 ECD (e.g., SEQ ID NO: 60), with a K_{D} of between about 1×10⁻⁸ M and about 1×10⁻¹⁰ M. In another embodiment, an anti-human LAG-3 antibody will have an affinity for human LAG-3 (e.g., SEQ ID NO: 57 or SEQ ID NO: 58), to a human LAG-3 ECD (e.g., SEQ ID NO: 59), and/or to a cynomolgus LAG3 ECD (e.g., SEQ ID NO: 60), with a Kp of between about 5×10⁻⁸ M and about 5×10⁻¹⁰ M. In another embodiment, an anti-human antibody will have an affinity for human LAG-3 (SEQ ID NO: 57 or SEQ ID NO: 58), for a human LAG-3 ECD (e.g., SEQ ID NO: 59), and/or for a cynomolgus LAG3 ECD (e.g., SEQ ID NO: 60), with a K_{D} of between about 1×10⁻⁹ M and about 1×10⁻¹⁰ M.

In another embodiment, the anti-human LAG-3 agonist antibody is a human IgG1 or IgG4 isotype. In another embodiment, the antibody is a human IgG1 isotype. A non-limiting example of a human IgG1 isotype of an anti-human LAG-3 agonist antibody is the amino acid sequence of SEQ ID NO: 11.

In another embodiment, the antigen binding domain of the anti-human LAG-3 is a single-chain variable fragment (scFv).

The present disclosure also provides a nucleic acid comprising a sequence encoding one or both of SEQ ID NO: 11 and SEQ ID NO: 12.

In another embodiment, the present disclosure provides a vector comprising a nucleic acid sequence encoding one or both of SEQ ID NO: 11 and SEQ ID NO: 12.

In another embodiment, the present disclosure provides a composition comprising a first vector comprising a nucleic acid sequence encoding SEQ ID NO: 11 and a second vector comprising a nucleic acid sequence encoding SEQ ID NO: 12.

In one embodiment, the present disclosure provides a cell comprising the vectors. In another embodiment, the present disclosure provides a cell comprising the composition. In another embodiment, the cell is a mammalian cell. In another embodiment, the cell or the mammalian cell is isolated. In another embodiment, the present disclosure provides a process of producing an antibody comprising culturing the cell under conditions such that the antibody is expressed and recovering the expressed antibody from the culture medium. In another embodiment, the present disclosure provides an antibody produced by culturing the cell under conditions such that the antibody is expressed and recovering the expressed antibody from the culture medium. In another embodiment, the present disclosure provides composition comprising the anti-human LAG-3 agonist antibody of the invention.

The present disclosure also provides a pharmaceutical composition comprising the anti-human LAG-3 agonist antibody of the invention, and a pharmaceutically acceptable excipient, diluent or carrier. In one p embodiment the pharmaceutical composition comprises arginine.

The present disclosure also provides a pre-filled syringe, a pharmaceutical composition comprising the anti-human LAG-3 agonist antibody of the invention, and a pharmaceutically acceptable excipient, diluent or carrier. In one embodiment the pharmaceutical composition comprises arginine.

The present disclosure also provides a method of treating autoimmune disease in a subject in need thereof, comprising administering to the subject a therapeutically effective amount of the anti-human LAG-3 agonist antibody of the invention. In one embodiment, the autoimmune disease is rheumatoid arthritis (RA), psoriasis (PsO), ulcerative colitis (UC), Type I diabetes mellitus (T1DM), lupus nephritis (LN) or systemic lupus erythematosus (SLE). In one embodiment, the autoimmune disease is active disease. In another embodiment, the autoimmune disease is in remission.

The present disclosure also provides the anti-human LAG-3 agonist antibody of the invention, for use in therapy.

The present disclosure also provides the anti-human LAG-3 agonist antibody of the invention, for use in the treatment of autoimmune disease. In one embodiment, the autoimmune disease is RA, PsO, UC, T1DM, LN or SLE. In another embodiment, the autoimmune disease is active disease. In another embodiment, the autoimmune disease is in remission.

The present disclosure also provides a pharmaceutical composition comprising the anti-human LAG-3 agonist antibody of the invention, for use in treating RA, PsO, UC, T1DM, LN or SLE. In one embodiment, the autoimmune disease is active disease. In another embodiment, the autoimmune disease is in remission.

The present disclosure also provides the use of the anti-human LAG-3 agonist antibody of the invention, in the manufacture of a medicament for the treatment of RA, PsO, UC, T1DM, LN or SLE.

Mutations are known to those of ordinary skill in the art that facilitate desired qualities. However, it cannot be predicted which, if any, or how many such mutations will facilitate the desired qualities of any particular antibody. The present invention provides antibodies that contain amino acid residue mutations that facilitate one or more of desired antibody expression, assembly, reduction or elimination Clq binding, reduction or elimination of non-specific and self-interaction, reduction of composition or formulation viscosity, elimination of deamidation residues, and reduction of immunogenicity.

In embodiments that refer to a method of treatment as described herein, such embodiments are also further embodiments for use in that treatment, or alternatively for the use in the manufacture of a medicament for use in that treatment.

In one embodiment, the anti-human LAG-3 agonist antibody of the invention is substantially pure. In another embodiment, the anti-human LAG-3 agonist antibody of the invention is sterile.

In one embodiment, the immune disease is RA, PsO, UC, T1DM, LN or SLE. In another embodiment, the immune disease is RA. In another embodiment, the immune disease is PsO. In another embodiment, the immune disease is UC. In another embodiment, the immune disease is T1DM. In another embodiment, the immune disease is LN. In another embodiment, the immune disease is SLE. In another embodiment, the immune disease is active disease. In another embodiment, the immune disease is in remission.

In another embodiment, an anti-human LAG-3 agonist antibody of the invention binds human LAG-3, but does not deplete T-cells.

In another embodiment, an anti-human LAG-3 agonist antibody of the invention agonizes the LAG-3 signaling pathway.

In another embodiment, an anti-human LAG-3 agonist antibody of the invention binds human LAG-3 with desirable association and dissociation rates for optimal agonist activity.

In another embodiment, an anti-human LAG-3 agonist antibody of the invention decreases T cell proliferation by promoting T-cell receptor signaling downregulation, instead of by depletion of T-cells.

In another embodiment, an anti-human LAG-3 agonist antibody of the invention agonizes human LAG-3 in an immunologically relevant context to achieve in vivo efficacy

The present disclosure also provides a method comprising: (a) contacting an anti-human LAG-3 agonist antibody of the invention with cells that express the extracellular domain of human LAG-3 fused to an enzyme donor subunit; (b) incubating the contacted cells under conditions suitable for the antibody to bind to the extracellular domain of human LAG-3; (c) contacting the cells with a reporter compound; (d) assaying the amount of reporter compound; and (e) determining an IC50 for the antibody binding to the cells. In one embodiment, the enzyme acceptor subunit of the reporter gene is beta-galactosidase, and the enzyme donor subunit is PK1 enzyme donor subunit of beta-galactosidase. In another embodiment, the antibody comprises the HC of SEQ ID NO: 11 and the LC of SEQ ID NO: 12.

### DETAILED DESCRIPTION

As used herein, the terms “a,” “an,” “the,” and similar terms used in the context of the present disclosure (especially in the context of the claims) are to be construed to cover both the singular and plural unless otherwise indicated herein or clearly contradicted by the context.

As used herein “LAG-3” refers to lymphocyte activation gene 3, also known as CD223, which belongs to the immunoglobulin superfamily. The terms LAG-3, LAG3, Lag-3 and Lag3 are synonymous.

As used herein “hLAG-3” or “human LAG-3” refers to a wild-type human LAG-3, preferably, a wild-type human LAG-3 that has the amino acid sequence set forth in SEQ ID NO: 57 (i.e., NCBI Reference Sequence NP_002277) or SEQ ID NO: 58.

Unless otherwise indicated herein, “LAG-3” refers to human LAG-3.

A LAG-3 polypeptide “extracellular domain” or “ECD” refers to a form of the LAG-3 polypeptide that is essentially free of the transmembrane and cytoplasmic domains. Preferably, a LAG-3 ECD has less than 1% of the transmembrane and cytoplasmic domain, more preferably, a LAG-3 ECD has less than 0.5% of such domains. Even more preferably, human LAG-3 ECD polypeptide is as shown in SEQ ID NO: 59. LAG-3 polypeptide ECD may prepared using methods known in the art. Alternatively, human LAG-3 polypeptide ECD may be purchased commercially from various vendors such as Bertyn Bioreagent (Rockville, MD, USA, Cat. No. 32083).

The term “antibody,” as used herein, refers to an immunoglobulin molecule that binds an antigen. Embodiments of an antibody include a monoclonal antibody, polyclonal antibody, human antibody, humanized antibody, chimeric antibody, bispecific or multispecific antibody, or conjugated antibody. The antibodies can be of any class (e.g., IgG, IgE, IgM, IgD, IgA), and any subclass (e.g., IgG1, IgG2, IgG3, IgG4). In one embodiment, the antibody or bispecific antibody of the invention is an IgG1.

An exemplary antibody of the present disclosure is an immunoglobulin G (IgG) type antibody comprised of four polypeptide chains: two heavy chains (HC) and two light chains (LC) that are cross-linked via inter-chain disulfide bonds. The amino-terminal portion of each of the four polypeptide chains includes a variable region of about 100-125 or more amino acids primarily responsible for antigen recognition. The carboxyl-terminal portion of each of the four polypeptide chains contains a constant region primarily responsible for effector function. Each HC is comprised of a VH and a heavy chain constant region. Each LC is comprised of a light chain variable region (VL) and a light chain constant region. The IgG isotype may be further divided into subclasses (e.g., IgG1, IgG2, IgG3, and IgG4).

The VH and VL regions can be further subdivided into regions of hyper-variability, termed complementarity determining regions (CDRs), interspersed with regions that are more conserved, termed framework regions (FR). The CDRs are exposed on the surface of the protein and are important regions of the antibody for antigen binding specificity. Each VH and VL is composed of three CDRs and four FRs, arranged from amino-terminus to carboxyl-terminus in the following order: FR1, CDR1, FR2, CDR2, FR3, CDR3, FR4. Herein, the three CDRs of the heavy chain are referred to as “HCDR1, HCDR2, and HCDR3” and the three CDRs of the LC are referred to as “LCDR1, LCDR2 and LCDR3”. The CDRs contain most of the residues that form specific interactions with the antigen. Assignment of amino acid residues to the CDRs may be done according to the well-known schemes, including those described in Kabat (Kabat et al., “Sequences of Proteins of Immunological Interest,” National Institutes of Health, Bethesda, Md. (1991)), Chothia (Chothia et al., “Canonical structures for the hypervariable regions of immunoglobulins”, Journal of Molecular Biology, 196, 901-917 (1987); Al-Lazikani et al., “Standard conformations for the canonical structures of immunoglobulins”, Journal of Molecular Biology, 273, 927-948 (1997)), North (North et al., “A New Clustering of Antibody CDR Loop Conformations”, Journal of Molecular Biology, 406, 228-256 (2011)), or IMGT (the international ImMunoGeneTics database available on at www.imgt.org; see Lefranc et al., Nucleic Acids Res. 1999; 27:209-212). In the disclosure herein, the Kabat method is used to assign amino acid residues to the CDRs.

Embodiments of the present disclosure also include antibody fragments (i.e., antigen binding domains or antigen-binding fragments) that, as used herein, comprise at least a portion of an antibody retaining the ability to specifically interact with an antigen or an epitope of the antigen, such as Fab, Fab′, F(ab′)₂, Fv fragments, scFv antibody fragments, scFab, disulfide-linked Fvs (sdFv), a Fd fragment.

The terms “antigen binding domain” and “antigen binding fragment” are used interchangeably herein, and as used herein, refer to a portion of an antibody that binds an antigen or an epitope of the antigen. For example, the antigen binding domain in the human LAG-3 agonist antibody of the invention binds to human LAG-3 (SEQ ID NO: 57 or SEQ ID NO: 58) or to a fragment thereof, e.g., LAG-3 ECD (SEQ ID NO: 59). More specifically, the antigen binding domain in the human LAG-3 agonist antibody of the invention binds to domain 2 (D2) of human LAG-3 (e.g., SEQ ID NO: 61) or regions within D2 (e.g., SEQ ID NO: 62, SEQ ID NO: 63, and/or SEQ ID NO: 64).

The term “agonize”, as used herein, refers to the ability of an antibody, antibody fragment, antigen binding domain, or a binding molecule to induce or increase one or more activities or functions associated with an antigen.

The present disclosure provides an anti-human LAG-3 antibody that binds Domain 2 of human LAG-3 (SEQ ID NO: 61), wherein the antibody binds an epitope comprising one or more amino acid residues within SPHHHLAESF (SEQ ID NO: 64) of human LAG-3 Domain 2 (SEQ ID NO: 61). In some embodiments the present disclosure provides an antibody that binds Domain 2 of human LAG-3 (SEQ ID NO: 61), wherein the antibody binds three or more amino acid resides within the PDRPASVHWFRNRGQGR VPVRESPHHHLAESF (SEQ ID NO: 62). In some embodiments, the epitope further comprises one or more amino acid residues within PQVSPMD (SEQ ID NO: 63) of human LAG-3 Domain 2.

The present disclosure provides an anti-human LAG-3 antibody that binds Domain 2 of human LAG-3 (SEQ ID NO: 61), wherein the antibody binds an epitope comprising one or more amino acid residues within SMTASPPGSLRASD (SEQ ID NO: 64). In some embodiments the present disclosure provides an anti-human LAG-3 antibody that binds Domain 2 of human LAG-3 (SEQ ID NO: 61), wherein the antibody is an agonist.

The term “Fc region” as used herein refers to a polypeptide comprising the CH2 and CH3 domains of a constant region of an immunoglobulin, e.g., IgG1, IgG2, IgG3, or IgG4. Optionally, the Fc region may include a portion of the hinge region or the entire hinge region of an immunoglobulin, e.g., IgG1, IgG2, IgG3, or IgG4. In some embodiments, the Fc region is a human IgG Fc region, e.g., a human IgG1 Fc region, human IgG2 Fc region, human IgG3 Fc region or human IgG4 Fc region. In some embodiments, the Fc region is a modified IgG Fc region with reduced or eliminated effector functions compared to the corresponding wild type IgG Fc region. The numbering of the residues in the Fc region is based on the EU index as described in Kabat (Kabat et al, Sequences of Proteins of Immunological Interest, 5th edition, Bethesda, MD: U.S. Dept. of Health and Human Services, Public Health Service, National Institutes of Health, 1991). The boundaries of the Fc region of an immunoglobulin HC might vary, and the human IgG heavy chain Fc region is usually defined as the stretch from the N-terminus of the CH2 domain (e.g., the amino acid residue at position 231 according to the EU index numbering) to the C-terminus of the CH3 domain (or the C-terminus of the immunoglobulin).

Preferably, antibodies of the present disclosure contain a Fc region which is a human IgG1 or IgG4 subtype. It is well-known that human IgG1 binds to the Fc-gamma receptor family (FcγR) as well as Clq. Interaction with these receptors can induce antibody-dependent cell cytotoxicity (ADCC) and complement-dependent cytotoxicity (CDC). Certain embodiments of the human LAG-3 agonist antibodies of the present invention contain an IgG1 Fc region or an Fc region derived from human IgG1, e.g., a modified IgG1 Fc region having altered Fc effector functions. In some embodiments of the human LAG-3 agonist antibodies of the present invention the IgG1 Fc region or an Fc region derived from human IgG1 comprises the well-known set of Fc region mutations L234A, L235A, and D265S (with reference to IMGT or EU Index numbering), which is commonly referred to as IgGIAAS or the like. The term “AAS” refers to L234A/L235A/D265S mutations. This is an example of effector null mutations in the Fc region (see, e.g., Pejchal et al., “Profiling the Biophysical Developability Properties of Common IgG1 Fc Effector Silencing Variants,” Antibodies, 12, 54 (2023). Other embodiments of the human LAG-3 agonist antibodies of the present invention contain an IgG4 Fc region or an Fc region derived from human IgG4, e.g., a modified IgG4 Fc region having altered Fc effector functions. For example, in some embodiments, a well-known serine to proline mutation at position 228 (“S228P” according to IMGT or EU numbering) is introduced into the IgG4 Fc region. This modified IgG4 Fc region is commonly referred to as IgG4P, IgG4 S228P, IgG4StoP, or the like. In one embodiment, the monoclonal anti-human LAG-3 agonist antibody of the invention does not exhibit substantial ADCC activity. In another embodiment, the monoclonal anti-human LAG-3 agonist antibody of the invention does not exhibit ADCC activity. In another embodiment, the anti-human LAG-3 agonist antibody of the invention does not exhibit substantial CDC activity. In another embodiment, the anti-human LAG-3 agonist antibody of the invention does not exhibit CDC activity.

In one embodiment, the monoclonal anti-human LAG-3 agonist antibody of the invention does not substantially deplete T cells. In another embodiment, the monoclonal anti-human LAG-3 agonist antibody of the invention does not deplete T cells.

An isolated DNA encoding a CH region can be converted to a full-length heavy chain gene by operably linking the VH-encoding DNA to another DNA molecule encoding heavy chain constant regions. The sequences of human, as well as other mammalian, heavy chain constant region genes are known in the art. DNA fragments encompassing these regions can be obtained, e.g., by standard PCR amplification.

An isolated DNA encoding a VL region may be converted to a full-length light chain gene by operably linking the VL-encoding DNA to another DNA molecule encoding a light chain constant region. The sequences of human, as well as other mammalian, light chain constant region genes are known in the art. DNA fragments encompassing these regions can be obtained by standard PCR amplification. The light chain constant region can be a kappa or lambda constant region. In one embodiment, the light chain constant region of an anti-LAG-3 agonist antibody of the invention is a kappa constant region.

The term “autoimmune disease” as used herein refers to undesirable conditions that arise from an inappropriate or unwanted immune reaction against self-cells and/or tissues or transplanted cells and/or tissues. The term “autoimmune disease” is meant to include such conditions, whether they be mediated by humoral or cellular immune responses. Exemplary autoimmune diseases or disorders include, but are not limited to, graft-versus-host disease (GVHD), solid organ transplantation rejection, vasculitis, LN, SLE, T1DM, multiple sclerosis (MS), giant cell arteritis (GCA), PsO, psoriatic arthritis (PsA), RA, inflammatory bowel disease (IBD), UC, ankylosing spondylitis (AS), Sjogren's syndrome (SjS), autoimmune hepatitis, scleroderma, celiac disease, Addison's disease, Hashimoto's disease, Graves' disease, atrophic gastritis/pernicious anemia, acquired hypogonadism/infertility, hypoparathyroidism, Coombs positive-hemolytic anemia, chronic allergic diseases (such as asthma, hay fever, or allergic rhinitis), Crohn's disease, male or female infertility, Behcet's, Wegener's granulomatosis, myocarditis, myositis, polymyalgia rheumatic (PMR), spontaneous abortion, vitiligo, atherosclerosis, autoimmune pancreatitis, bullous pemphigoid, chronic viral infections, and myasthenia gravis. For purposes of the present disclosure, autoimmune diseases are GVHD, solid organ transplantation rejection, vasculitis, SLE, T1DM, MS, GCA, PsO, PsA, RA, IBD, UC, AS, SjS, autoimmune hepatitis, and scleroderma.

The terms “nucleic acid” or “polynucleotide”, as used interchangeably herein, refer to polymers of nucleotides, including single-stranded and/or double-stranded nucleotide-containing molecules, such as DNA, cDNA and RNA molecules, incorporating native, modified, and/or analogs of, nucleotides. Polynucleotides of the present disclosure may also include substrates incorporated therein, for example, by DNA or RNA polymerase or a synthetic reaction.

The polynucleotides of the present disclosure can be expressed in a host cell after the sequences have been operably linked to an expression control sequence. The expression vectors are typically replicable in the host organisms either as episomes or as an integral part of the host chromosomal DNA. Commonly, expression vectors will contain selection markers, e.g., tetracycline, neomycin, and dihydrofolate reductase, to permit detection of those cells transformed with the desired DNA sequences.

The antibodies of the present disclosure can readily be produced in mammalian cells, non-limiting examples of which includes CHO, NSO, HEK293 or COS cells. The host cells are cultured using techniques well known in the art.

The vectors containing the polynucleotide sequences of interest (e.g., the polynucleotides encoding the polypeptides of the antibody and expression control sequences) can be transferred into the host cell by well-known methods, which vary depending on the type of cellular host.

Various methods of protein purification may be employed to purify proteins, including, but not limited to, antibodies, and such methods are known in the art.

In other embodiments of the present disclosure, cells, antibodies, or the nucleic acids encoding the same, are provided in isolated form. As used herein, the term “isolated” refers to a cell, protein, peptide, or nucleic acid which is free or substantially free from any other macromolecular species found in a cellular environment. “Substantially free” as used herein means the protein, peptide, or nucleic acid of interest comprises more than 80% (on a molar basis) of the macromolecular species present, preferably more than 90%, and more preferably more than 95%.

An antibody of the present disclosure, or a pharmaceutical composition comprising the same, may be administered by parenteral routes, non-limiting examples of which are subcutaneous administration and intravenous administration. An antibody of the present disclosure may be administered to a patient alone with pharmaceutically acceptable carriers, diluents, or excipients in single or multiple doses. Pharmaceutical compositions of the present disclosure can be prepared by methods well known in the art (e.g., Remington: The Science and Practice of Pharmacy, 22nd ed. (2012), A. Loyd et al., Pharmaceutical Press) and comprise an antibody, as disclosed herein, and one or more pharmaceutically acceptable carriers, diluents, or excipients.

“Binds to human LAG-3” as used herein, in reference to the affinity of an anti-human LAG-3 agonist antibody to a human LAG-3 (e.g., SEQ ID NO: 57 or SEQ ID NO: 58), to a human LAG-3 ECD (e.g., SEQ ID NO: 59), and/or to a cynomolgus LAG3 ECD (e.g., SEQ ID NO: 60), is intended to mean, unless indicated otherwise, a K_{D} of about 1×10⁻⁷ M, of about 1×10⁻⁸ M, of about 1×10⁻⁹ M, of about 5×10⁻¹⁰ M, of about 1×10⁻¹⁰ M, or of about 5×10⁻¹¹ M, as determined by methods known in the art and/or by methods essentially as described herein, including, but not limited to, use of a surface plasmon resonance (SPR) biosensor at 25° C. or 37° C. In another embodiment, an anti-human LAG-3 agonist antibody of the present disclosure binds human LAG-3 (e.g., SEQ ID NO: 57 or SEQ ID NO: 58), to a human LAG-3 ECD (e.g., SEQ ID NO: 59), and/or to a cynomolgus LAG3 ECD (e.g., SEQ ID NO: 60), with a K_{D} of between about 1×10⁻⁸ M and about 5×10⁻¹⁰ M as determined by methods known in the art and/or by methods essentially as described herein, including by use of a SPR biosensor at 25° C. or 37° C. In another embodiment, an anti-human LAG-3 antibody will have an affinity for human LAG-3 (e.g., SEQ ID NO: 57 or SEQ ID NO: 58), for human LAG-3 ECD (e.g., SEQ ID NO: 59), and/or for cynomolgus LAG3 ECD (e.g., SEQ ID NO: 60), of between about 5×10⁻⁸ M and about 5×10⁻¹⁰ M as determined by methods known in the art and/or by methods essentially as described herein, including by use of a SPR biosensor at 25° C. or 37° C. In another embodiment, an anti-human LAG-3 antibody will have Kon and Koff values for human LAG-3 ECD (e.g., SEQ ID NO: 59) of about 1.0×10⁻⁴ to about 1.0×10⁻³ and about 1.3×10⁵ to about 2.0×10⁵, respectively (as determined by SPR using on BIAcore®8K essentially as described herein).

The terms “selectively binds” or “specifically binds” mean that an antibody of the invention interacts more frequently, more rapidly, with greater duration, with greater affinity, or with some combination of the above to human and/or cynomolgus LAG-3 than do other substances. In one embodiment, “specifically binds” means that an antibody of the invention binds to human LAG-3, with a K_{D} of about 0.1 mM or less. In another embodiment, “specifically binds” means that an antibody of the invention binds to human LAG-3 with a K_{D} of about 0.01 mM or less. In another embodiment, “specifically binds” means that an antibody of the invention binds to human LAG-3 with a K_{D} of about 0.001 mM or less. In another embodiment, “specifically binds” means that an antibody of the invention binds to human LAG-3 with a K_{D} of about 0.0001 mM or less.

The terms “bind” or “binds” as used herein are intended to mean, unless indicated otherwise, the ability of a protein or molecule to form a chemical bond or attractive interaction with another protein or molecule, which results in proximity of the two proteins or molecules as determined by common methods known in the art.

As referred to herein, the term “epitope” refers to the amino acid residues, of an antigen, that are bound by an antibody. An epitope can be a linear epitope, a conformational epitope (or discontinuous), or a hybrid epitope.

The term “epitope” may be used in reference to a structural epitope. A structural epitope, according to some embodiments, may be used to describe the region of an antigen which is covered by an antibody (e.g., an antibody's footprint when bound to the antigen). In some embodiments, a structural epitope may describe the amino acid residues of the antigen that are within a specified proximity (e.g., within a specified number of Angstroms) of an amino acid residue of the antibody.

The term “epitope” may also be used in reference to a functional epitope. A functional epitope, according to some embodiments, may be used to describe amino acid residues of the antigen that interact with amino acid residues of the antibody in a manner contributing to the binding energy between the antigen and the antibody.

An epitope can be determined according to different experimental techniques, also called “epitope mapping techniques.” It is understood that the determination of an epitope may vary based on the different epitope mapping techniques used and may also vary with the different experimental conditions used, e.g., due to the conformational changes or cleavages of the antigen induced by specific experimental conditions. Epitope mapping techniques are known in the art (e.g., Rockberg and Nilvebrant, Epitope Mapping Protocols: Methods in Molecular Biology, Humana Press, 3rd ed. 2018), including but not limited to, X-ray crystallography, nuclear magnetic resonance (NMR) spectroscopy, site-directed mutagenesis, species swap mutagenesis, alanine-scanning mutagenesis, hydrogen-deuterium exchange (HDX) and cross-blocking assays.

As used herein, the term “competes for binding” or “competes with”, refers to two antibodies which cross-compete (i.e., compete against each other) for binding to the same antigen. In some embodiments, two antibodies may compete for binding to the same antigen where they bind to spatially overlapping regions of the same antigen. In some embodiments, two antibodies may compete for binding to a same antigen where the antibodies bind to non-overlapping regions of the antigen, but the binding of one antibody blocks binding by the other antibody, for example, due to steric hindrance or conformational changes of the antigen induced by the first antibody. The term “substantially pure” as used herein refers to material, e.g., an antibody of the invention, that is at least 50%, 60%, 70%, 80%, 90%, 95%, 96%, 97%, 98%, 99% or 100% free of contaminants.

As used herein the term “about” implies plus or minus ten percent of the stated value or range of values. For example: “about” 12, includes values ranging from 10.8 (inclusive) to 13.2 (inclusive); about 10 wt. percent encompasses formation that include between 9 (inclusive) to 11 (inclusive) wt. percent; and the like.

As used herein, the term “adaptive immunity” includes the arm of the immune response which, in contrast to the innate arm of the immune response is antigen specific and shows enhanced, secondary antigen-specific immune responses upon re-stimulation with the same antigen.

The term “treating” (or “treat” or “treatment”) refers to slowing, interrupting, arresting, alleviating, stopping, reducing, or reversing the progression or severity of an existing symptom, disorder, condition, or disease.

“Effective amount” means the amount of an antibody of the invention or pharmaceutical composition comprising such an antibody that will elicit the biological or medical response of or desired therapeutic effect on a tissue, system, animal, mammal or human that is being sought by the researcher, medical doctor, or other clinician. An effective amount of the antibody may vary according to factors such as the disease state, age, sex, and weight of the individual, and the ability of the antibody to elicit a desired response in the individual. An effective amount is also one in which any toxic or detrimental effect of the antibody is outweighed by the therapeutically beneficial effects. Such benefit includes any one or more of: an increased immune tolerance of transplanted organs; stabilized autoimmune disease or disorder; or improving signs or symptoms of an autoimmune disorder, etc. An effective amount can be readily determined by one skilled in the art, by the use of known techniques, and by observing results obtained under analogous circumstances. In determining the effective amount for a patient, a number of factors are considered by the attending diagnostician, including, but not limited to: the patient's size, age, and general health; the specific disease or disorder involved; the degree of, or involvement, or the severity of the disease or disorder; the response of the individual patient; the particular compound administered; the mode of administration; the bioavailability characteristics of the preparation administered; the dose regimen selected; the use of concomitant medication; and other relevant circumstances.

Dosage regimens for administering antibodies of the invention may be adjusted to provide the optimum desired response (e.g., a therapeutic effect).

A potential advantage of methods disclosed herein is the possibility of producing marked and/or prolonged relief in a patient suffering from an autoimmune disorder with an acceptable safety profile including acceptable tolerability, toxicities and/or adverse events, so that the patient benefits from the treatment method overall. The efficacy of the treatment of the present disclosure can be measured by various endpoints that are commonly used in evaluating treatments for various autoimmune disorders including, but not limited to, American College of Rheumatology (ACR) 20, ACR50, ACR70, Psoriasis Area and Severity Index (PASI) 50, PASI75, PASI90, PASI100, Systemic Lupus Erythmatosus Disease Activity Index (SLEDAI). Various other approaches to determining efficacy of any particular therapy of the present disclosure can be optionally employed, including, for example, immune cell activation markers, measures of inflammation, cell-cycle dependent biomarkers measurement visualization, and/or measurement of response through pain assessments.

The term “modified human IgG1” as used herein means a human IgG1 engineered to modify the binding of the human IgG1 to at least one human Fc gamma receptor. Typically, this is performed by mutating residues that lead to a reduction in the binding of the antibody to the Fc gamma receptor(s), e.g., P329A, L234A and L235 A mutations.

The present disclosure provides an antibody, or an antigen binding domain thereof, that binds to human LAG-3, wherein the antibody comprises a heavy chain variable region (VH) and a light chain variable region (VL), wherein the VH comprises heavy chain complementarity determining regions (HCDR) HCDR1, HCDR2, and HCDR3, and the VL comprises light chain complementarity determining regions (LCDR) LCDR1, LCDR2, and LCDR3, wherein:
HCDR1 comprises SEQ ID NO: 65, HCDR2 comprises SEQ ID NO: 2, and HCDR3 comprises SEQ ID NO: 48;
HCDR1 comprises SEQ ID NO: 1, HCDR2 comprises SEQ ID NO: 2, and HCDR3 comprises SEQ ID NO: 48;
HCDR1 comprises SEQ ID NO: 1, HCDR2 comprises SEQ ID NO: 2, and HCDR3 comprises SEQ ID NO: 3;
HCDR1 comprises SEQ ID NO: 1, HCDR2 comprises SEQ ID NO: 2, and HCDR3 comprises SEQ ID NO: 15;
HCDR1 comprises SEQ ID NO: 52, HCDR2 comprises SEQ ID NO: 22, and HCDR3 comprises SEQ ID NO: 53;
HCDR1 comprises SEQ ID NO: 21, HCDR2 comprises SEQ ID NO: 22, and HCDR3 comprises SEQ ID NO: 23;
HCDR1 comprises SEQ ID NO: 31, HCDR2 comprises SEQ ID NO: 22, and HCDR3 comprises SEQ ID NO: 32; or
HCDR1 comprises SEQ ID NO: 38, HCDR2 comprises SEQ ID NO: 39, and HCDR3 comprises SEQ ID NO: 40; and
LCDR1 comprises SEQ ID NO: 66, LCDR2 comprises SEQ ID NO: 67, and LCDR3 comprises SEQ ID NO: 68;
LCDR1 comprises SEQ ID NO: 49, LCDR2 comprises SEQ ID NO: 5, and LCDR3 comprises SEQ ID NO: 6;
LCDR1 comprises SEQ ID NO: 4, LCDR2 comprises SEQ ID NO: 5, and LCDR3 comprises SEQ ID NO: 6;
LCDR1 comprises SEQ ID NO: 16, LCDR2 comprises SEQ ID NO: 5, and LCDR3 comprises SEQ ID NO: 6;
LCDR1 comprises SEQ ID NO: 24, LCDR2 comprises SEQ ID NO: 25, and LCDR3 comprises SEQ ID NO: 54;
LCDR1 comprises SEQ ID NO: 24, LCDR2 comprises SEQ ID NO: 25, and LCDR3 comprises SEQ ID NO: 26;
LCDR1 comprises SEQ ID NO: 24, LCDR2 comprises SEQ ID NO: 25, and LCDR3 comprises SEQ ID NO: 33; or
LCDR1 comprises SEQ ID NO: 41, LCDR2 comprises SEQ ID NO: 42, and LCDR3 comprises SEQ ID NO: 43. In some embodiments, the anti-human LAG-3 antibody of the invention is a human LAG-3 agonist antibody.

The present disclosure provides an antibody, or antigen binding domain thereof, that binds to human LAG-3, wherein the antibody comprises a heavy chain variable region (VH) and a light chain variable region (VL),
wherein the VH comprises a VH selected from the group consisting of:
   SEQ ID NO: 69, SEQ ID NO: 50, SEQ ID NO: 7, SEQ ID NO: 17, SEQ ID NO: 55, SEQ ID NO: 27, SEQ ID NO: 34 and SEQ ID NO: 44; and
wherein the VL comprises a VL selected from the group consisting of:
   SEQ ID NO: 70, SEQ ID NO: 51, SEQ ID NO: 8, SEQ ID NO: 18, SEQ ID NO: 56, SEQ ID NO: 28, SEQ ID NO: 35, and SEQ ID NO: 45. In certain embodiments, the VH comprises SEQ ID NO: 69 and the VL comprises SEQ ID NO: 70, the VH comprises SEQ ID NO: 50 and the VL comprises SEQ ID NO: 51, the VH comprises SEQ ID NO: 7 and the VL comprises SEQ ID NO: 8, the VH comprises SEQ ID NO: 17 and the VL comprises SEQ ID NO: 18, the VH comprises SEQ ID NO: 55 and the VL comprises SEQ ID NO: 56, the VH comprises SEQ ID NO: 27 and the VL comprises SEQ ID NO: 28, the VH comprises SEQ ID NO: 34 and the VL comprises SEQ ID NO: 35, or the VH comprises SEQ ID NO: 44 and the VL comprises SEQ ID NO: 45. In some embodiments, the anti-human LAG-3 antibody comprises a constant heavy (CH) region comprising SEQ ID NO: 9 and a constant light (CL) region comprising SEQ ID NO: 10. In some embodiments, the anti-human LAG-3 antibody of the invention is a human LAG-3 agonist antibody. In some embodiments, the anti-human LAG-3 antibody is a IgG1 or IgG4 subtype or a variant thereof such as an IgGIAAS or an IgG4P.

The present disclosure provides an antibody, or an antigen binding domain thereof, that binds to human LAG-3, wherein the antibody comprises:
a. a HC comprising SEQ ID NO: 71 and a LC comprising SEQ ID NO: 72;
b. a HC comprising SEQ ID NO: 11 and a LC comprising SEQ ID NO: 12;
c. a HC comprising SEQ ID NO: 19 and a LC comprising SEQ ID NO: 20;
d. a HC comprising SEQ ID NO: 29 and a LC comprising SEQ ID NO: 30;
e. a HC comprising SEQ ID NO: 36 and a LC comprising SEQ ID NO: 37; or
f. a HC comprising SEQ ID NO: 46 and a LC comprising SEQ ID NO: 47.

In another embodiment, an anti-human LAG-3 antibody of the invention is a LAG-3 agonist antibody.

In some embodiments, an anti-human LAG-3 antibody of present invention provides an antibody, or an antigen binding domain thereof, that binds to human LAG-3, wherein the antibody comprises:
a. a HC comprising a sequence having at least 95% sequence identity to SEQ ID NO: 71 and a LC comprising a sequence having at least 95% sequence identity to SEQ ID NO: 72;
b. a HC comprising a sequence having at least 95% sequence identity to SEQ ID NO: 11 and a LC comprising a sequence having at least 95% sequence identity to SEQ ID NO: 12;
c. a HC comprising a sequence having at least 95% sequence identity to SEQ ID NO: 19 and a LC comprising a sequence having at least 95% sequence identity to SEQ ID NO: 20;
d. a HC comprising a sequence having at least 95% sequence identity to SEQ ID NO: 29 and a LC comprising a sequence having at least 95% sequence identity to SEQ ID NO: 30;
e. a HC comprising a sequence having at least 95% sequence identity to SEQ ID NO: 36 and a LC comprising a sequence having at least 95% sequence identity to SEQ ID NO: 37; or
f. a HC comprising a sequence having at least 95% sequence identity to SEQ ID NO: 46 and a LC comprising a sequence having at least 95% sequence identity to SEQ ID NO: 47.

In some embodiments, an anti-human LAG-3 antibody of present invention provides an antibody, or an antigen binding domain thereof, that binds to human LAG-3, wherein the antibody comprises:
a. a HC comprising a sequence having at least 90% sequence identity to SEQ ID NO: 71 and a light chain comprising a sequence having at least 90% sequence identity to SEQ ID NO: 72;
b. a HC comprising a sequence having at least 90% sequence identity to SEQ ID NO: 11 and a LC comprising a sequence having at least 90% sequence identity to SEQ ID NO: 12;
c. a HC comprising a sequence having at least 90% sequence identity to SEQ ID NO: 19 and a light chain comprising a sequence having at least 90% sequence identity to SEQ ID NO: 20;
d. a HC comprising a sequence having at least 90% sequence identity to SEQ ID NO: 29 and a light chain comprising a sequence having at least 90% sequence identity to SEQ ID NO: 30;
e. a HC comprising a sequence having at least 90% sequence identity to SEQ ID NO: 36 and a light chain comprising a sequence having at least 90% sequence identity to SEQ ID NO: 37; or
f. a HC comprising a sequence having at least 90% sequence identity to SEQ ID NO: 46 and a light chain comprising a sequence having at least 90% sequence identity to SEQ ID NO: 47.

In some embodiments, the antibody of the present invention includes two HC and two light LC, wherein the HC and LC polypeptides are selected from the group consisting of:
a. HC of SEQ ID NO: 71 and LC of SEQ ID NO: 72;
b. HC of SEQ ID NO: 11 and LC of SEQ ID NO: 12;
c. HC of SEQ ID NO: 19 and LC of SEQ ID NO: 20;
d. HC of SEQ ID NO: 29 and LC of SEQ ID NO: 30;
e. HC of SEQ ID NO: 36 and LC of SEQ ID NO: 37; and
f. HC of SEQ ID NO: 46 and LC of SEQ ID NO: 47.

In another embodiment, an anti-human LAG-3 antibody of the invention binds to human LAG-3 (e.g., SEQ ID NO: 57 or SEQ ID NO: 58), to a human LAG-3 ECD (e.g., SEQ ID NO: 59), and/or to a cynomolgus LAG3 ECD (e.g., SEQ ID NO: 60), with a K_{D} of about 1×10⁻⁷ M to about 5×10⁻¹¹ M as determined by methods known in the art and/or by methods essentially as described herein, including, but not limited to, use of a surface plasmon resonance (SPR) biosensor at 25° C. or 37° C. In another embodiment, an anti-human LAG-3 agonist antibody of the present disclosure binds to human LAG-3 (e.g., SEQ ID NO: 57 or SEQ ID NO: 58), to a human LAG-3 ECD (e.g., SEQ ID NO: 59), and/or to a cynomolgus LAG3 ECD (e.g., SEQ ID NO: 60), with a K_{D} of between about 1×10⁻⁸ M and about 1×10⁻¹⁰ M. In another embodiment, an anti-human LAG-3 antibody will have an affinity for human LAG-3 (e.g., SEQ ID NO: 57 or SEQ ID NO: 58), for a human LAG-3 ECD (e.g., SEQ ID NO: 59), and/or for a cynomolgus LAG3 ECD (e.g., SEQ ID NO: 60), with a K_{D} of between about 5×10⁻⁸ M and about 5×10⁻¹⁰ M. In another embodiment, an anti-human antibody will have an affinity for human LAG-3 (e.g., SEQ ID NO: 57 or SEQ ID NO: 58), a human LAG-3 ECD (e.g., SEQ ID NO: 59), and/or a cynomolgus LAG3 ECD (e.g., SEQ ID NO: 60), with a K_{D} of between about 1×10⁻⁹ M and about 1×10⁻¹⁰ M.

In another embodiment, the anti-human LAG-3 agonist antibody is a human IgG1 or IgG4 isotype. In another embodiment, the antibody is a human IgG1 isotype. A non-limiting example of a human IgG1 isotype of an anti-human LAG-3 agonist antibody is the amino acid sequence of SEQ ID NO: 11.

In another embodiment, the antigen binding domain of the anti-human LAG-3 is a single-chain variable fragment (scFv).

The present disclosure also provides a nucleic acid comprising a sequence encoding one or both of SEQ ID NO: 11 and SEQ ID NO: 12.

In another embodiment, the present disclosure provides a vector comprising a nucleic acid sequence encoding one or both of SEQ ID NO: 11 and SEQ ID NO: 12.

In another embodiment, the present disclosure provides a composition comprising a first vector comprising a nucleic acid sequence encoding SEQ ID NO: 11 and a second vector comprising a nucleic acid sequence encoding SEQ ID NO: 12.

In one embodiment, the present disclosure provides a cell comprising the vectors. In another embodiment, the present disclosure provides a cell comprising the composition. In another embodiment, the cell is a mammalian cell. In another embodiment, the cell or the mammalian cell is isolated. In another embodiment, the present disclosure provides a process of producing an antibody comprising culturing the cell under conditions such that the antibody is expressed and recovering the expressed antibody from the culture medium. In another embodiment, the present disclosure provides an antibody produced by culturing the cell under conditions such that the antibody is expressed and recovering the expressed antibody from the culture medium. In another embodiment, the present disclosure provides composition comprising the anti-human LAG-3 agonist antibody of the invention.

The present disclosure also provides a pharmaceutical composition comprising the anti-human LAG-3 agonist antibody of the invention, and a pharmaceutically acceptable excipient, diluent or carrier. In one embodiment the pharmaceutical composition comprises arginine.

The present disclosure also provides a pre-filled syringe, a pharmaceutical composition comprising the anti-human LAG-3 agonist antibody of the invention, and a pharmaceutically acceptable excipient, diluent or carrier. In one embodiment the pharmaceutical composition comprises arginine.

The present disclosure also provides a method of treating autoimmune disease in a subject in need thereof, comprising administering to the subject a therapeutically effective amount of the anti-human LAG-3 agonist antibody of the invention. In one embodiment, the autoimmune disease is RA, PsO, UC, T1DM, LN, or SLE. In one embodiment, the autoimmune disease is active disease. In another embodiment, the autoimmune disease is in remission.

The present disclosure also provides the anti-human LAG-3 agonist antibody of the invention, for use in therapy.

The present disclosure also provides the anti-human LAG-3 agonist antibody of the invention, for use in the treatment of autoimmune disease. In one embodiment, the autoimmune disease is RA, PsO, UC, T1DM, LN, or SLE. In another embodiment, the autoimmune disease is active disease. In another embodiment, the autoimmune disease is in remission.

The present disclosure also provides a pharmaceutical composition comprising the anti-human LAG-3 agonist antibody of the invention, for use in treating autoimmune diseases such as RA, PsO, UC, T1DM, LN, or SLE. In one embodiment, the autoimmune disease is active disease. In another embodiment, the immune disease is in remission.

The present disclosure also provides the use of the anti-human LAG-3 agonist antibody of the invention, in the manufacture of a medicament for the treatment of RA, PsO, UC, T1DM, LN, or SLE.

Mutations are known to those of ordinary skill in the art that facilitate desired qualities. However, it cannot be predicted which, if any, or how many such mutations will facilitate the desired qualities of any particular antibody. The present invention provides antibodies that contain amino acid residue mutations that facilitate one or more of desired antibody expression, assembly, reduction or elimination Clq binding, reduction or elimination of non-specific and self-interaction, reduction of composition or formulation viscosity, elimination of deamidation residues, and reduction of immunogenicity.

In embodiments that refer to a method of treatment as described herein, such embodiments are also further embodiments for use in that treatment, or alternatively for the use in the manufacture of a medicament for use in that treatment.

In one embodiment, the anti-human LAG-3 agonist antibody of the invention is substantially pure. In another embodiment, the anti-human LAG-3 agonist antibody of the invention is sterile.

In one embodiment, the autoimmune disease is RA, PsO, UC, T1DM, LN, or SLE. In another embodiment, the autoimmune disease is rheumatoid arthritis. In another embodiment, the autoimmune disease is ulcerative colitis. In another embodiment, the autoimmune disease is Type I diabetes mellitus. In another embodiment, the autoimmune disease is lupus nephritis. In another embodiment, the autoimmune disease is systemic lupus erythematosus. In another embodiment, the immune disease is active disease. In another embodiment, the immune disease is in remission.

In another embodiment, an anti-human LAG-3 agonist antibody of the invention binds human LAG-3, but does not deplete T-cells.

In another embodiment, an anti-human LAG-3 agonist antibody of the invention agonizes the LAG-3 signaling pathway.

In another embodiment, an anti-human LAG-3 agonist antibody of the invention binds human LAG-3 with desirable association and dissociation rates for optimal agonist activity.

In another embodiment, an anti-human LAG-3 agonist antibody of the invention decreases T cell proliferation by promoting T-cell receptor signaling downregulation, instead of by depletion of T-cells.

In another embodiment, an anti-human LAG-3 agonist antibody of the invention agonizes human LAG-3 in an immunologically relevant context to achieve in vivo efficacy

The present disclosure also provides a method comprising: (a) contacting an anti-human LAG-3 agonist antibody of the invention with cells that express the extracellular domain of human LAG-3 fused to an enzyme donor subunit; (b) incubating the contacted cells under conditions suitable for the antibody to bind to the extracellular domain of human LAG-3; (c) contacting the cells with a reporter compound; (d) assaying the amount of reporter compound; and (e) determining an IC50 for the antibody binding to the cells. In one embodiment, the enzyme acceptor subunit of the reporter gene is beta-galactosidase, and the enzyme donor subunit is PK1 enzyme donor subunit of beta-galactosidase. In another embodiment, the antibody comprises the HC of SEQ ID NO: 11 and the LC of SEQ ID NO: 12.

Methods for assaying LAG-3 activity in vitro are known to those of ordinary skill in the art, for example, in Angin M, et al., J. Immunol. 2020; 204 (4): 810-818.

In vivo murine models of immune activity are well known to those of ordinary skill in the art, as shown herein, and as disclosed, e.g., Vincelette J, et al., Arthritis Res Ther. 2007; 9 (6): R123. doi: 10.1186/ar2331.

A DNA molecule of the present disclosure is a DNA molecule that comprises a non-naturally occurring polynucleotide sequence encoding a polypeptide having the amino acid sequence of at least one of the polypeptides in an antibody of the present disclosure.

The polynucleotides of the present disclosure may be expressed in a host cell after the sequences are operably linked to an expression control sequence. The expression vectors are typically replicable in the host organisms either as episomes or as an integral part of the host chromosomal DNA. Commonly, expression vectors contain selection markers, e.g., tetracycline, neomycin, and dihydrofolate reductase, to permit detection of those cells transformed with the desired DNA sequences.

An expression vector containing the polynucleotide sequences of interest (e.g., the polynucleotides encoding the polypeptides of an antibody of the invention and expression control sequences) can be transferred into a host cell by known methods, which vary depending on the type of host cells. An host cell (e.g., a mammalian cell) includes cells stably or transiently transfected, transformed, transduced, or infected with one or more expression vectors expressing all or a portion of the anti-human LAG-3 agonist antibody described herein. According to some embodiments, a host cell may be stably or transiently transfected, transformed, transduced, or infected with an expression vector expressing HC polypeptides and an expression vector expressing LC polypeptides of the anti-human LAG-3 agonist antibodies, or antigen binding domains thereof, as described herein. In some embodiments, a host cell may be stably or transiently transfected, transformed, transduced, or infected with an expression vector expressing HC and LC polypeptides of the anti-human LAG-3 agonist antibody, or antigen binding domain thereof, described herein. An antibody, or antigen binding domain thereof, of the present disclosure may readily be produced in mammalian host cells, non-limiting examples of which includes CHO, NSO, HEK293 or COS cells. The host cells may be cultured using techniques known in the art.

Mammalian expression of antibodies typically results in glycosylation. Glycosylation of antibodies is typically either N-linked or O-linked. N-linked glycosylation refers to the attachment of a carbohydrate moiety to the side chain of an asparagine residue. O-linked glycosylation refers to the attachment of a sugar, for example N-acetylgalactosamine, galactose, or xylose to a hydroxyamino acid. Typically, glycosylation occurs in the Fc region of the antibody at a highly conserved N-glycosylation site (e.g., position 297 in IgG1, according to IMGT or EU Index numbering). Glycosylation sites can be modified to alter glycosylation (e.g., blocking or reducing glycosylation or altering the amino acid sequence to produce additional or diverse glycosylation).

Mammalian expression of antibodies from IgG subclasses can result in clipping of C-terminal amino acids from one or both heavy chains; for example, one or two C-terminal amino acids can be removed for IgG1 antibodies. For IgG1 antibodies, if a C-terminal lysine is present, then it may be truncated or clipped off from the heavy chain during expression. Additionally, a penultimate glycine may also be truncated or clipped off (e.g., see SEQ ID NO: from the heavy chain as well.

Mammalian expression of antibodies can also result in the modification of N-terminal amino acids. For example, where the N-terminal most amino acid of a HC or LC is a glutamine, it may be modified into pyro-glutamic acid.

Various methods of protein purification may be employed to purify an antibody of the present disclosure and such methods are known in the art and described, for example, in Deutscher, Methods in Enzymology 182:83-89 (1990) and Scopes, Protein Purification: Principles and Practice, 3rd Edition, Springer, NY (1994).

Sequences referred to herein are numbered according to the sequence identifier numbers listed in Table 1. The sequences in Table 1 are amino acid sequences, unless otherwise indicated.

TABLE 1
Examples of LAG-3 Antibodies and Sequences Related Thereto

| | Antibody | Antibody | Antibody | Antibody | Antibody |
|---|---|---|---|---|---|
| | A | B | C | D | E |
| HCDR1 | 1 | 1 | 21 | 31 | 38 |
| HCDR2 | 2 | 2 | 22 | 22 | 39 |
| HCDR3 | 3 | 15 | 23 | 32 | 40 |
| LCDR1 | 4 | 16 | 24 | 24 | 41 |
| LCDR2 | 5 | 5 | 25 | 25 | 42 |
| LCDR3 | 6 | 6 | 26 | 33 | 43 |
| VH | 7 | 17 | 27 | 34 | 44 |
| VL | 8 | 18 | 28 | 35 | 45 |
| CH Constant | 9 | | | | |
| CL Constant | 10 | | | | |
| HC | 11 | 19 | 29 | 36 | 46 |
| LC | 12 | 20 | 30 | 37 | 47 |
| HC [DNA] | 13 | | | | |
| LC [DNA] | 14 | | | | |

### Examples

### Antibody Expression and Purification

The anti-human LAG-3 agonist antibodies of the invention may be expressed and purified essentially as follows. An appropriate host cell, such as HEK 293 or CHO, may be either transiently or stably transfected with an expression system for secreting antibodies using an optimal predetermined heavy chain:light chain vector ratio or a single vector system encoding both HC and light chain. The antibody of the present disclosure may be either transiently or stably transfected with an expression system for secreting antibodies using one or more DNA molecules encoding for a HC and a LC for a LAG-3 agonist antibody of the invention.

The antibodies may be purified using one of many commonly-used techniques. For example, the medium may be conveniently applied to a MabSelect column (GE Healthcare), or KappaSelect column (GE Healthcare), that has been equilibrated with a compatible buffer, such as phosphate buffered saline (pH 7.4). The column may be washed to remove nonspecific binding components. The bound antibody may be eluted, for example, by pH gradient (such as 20 mM Tris buffer pH 7.0 to 10 mM sodium citrate buffer pH 3.0, or phosphate buffered saline pH 7.4 to 100 mM glycine buffer pH 3.0). Antibody fractions may be detected, such as by UV absorbance or SDS-PAGE, and then may be pooled. Further purification is optional, depending on the intended use. The purified antibody may be concentrated and/or sterile filtered using common techniques. Soluble aggregate, multimers and mispairings may be effectively removed by common techniques, including size exclusion, hydrophobic interaction, ion exchange, multimodal, affinity or hydroxyapatite chromatography. The purified antibody may be immediately frozen at −70° C. or may be lyophilized.

### Binding to Human or Cynomolgus Monkey LAG-3

Surface Plasmon Resonance (SPR) at 37° C. is performed to determine the binding kinetics and affinity of LAG-3 antibodies to human LAG-3 and/or cynomolgus monkey LAG-3. A Biacore® T200 (Cytiva, Marlborough, MA) was used to measure the binding kinetics and affinities of Antibody A to human LAG-3 soluble extracellular domain (ECD) SEQ ID NO: 59) and to cynomolgus LAG-3 ECD (ACROBiosystems, Cat #LA3-C82H3; SEQ ID NO: 59) by surface plasmon resonance. Samples were diluted in HBS-EP+ (10 mM Hepes, 150 mM NaCl, 3 mM EDTA, 0.05% Tween-20, pH 7.6) (Teknova Cat #H8022) with 5 g/L of BSA (Jackson ImmunoResearch Cat #001-000-161) running buffer. PrismA Series S Sensor chip (Cat #29650263) is purchased from Cytiva.

Binding was evaluated using multi-cycle kinetics by an antibody capture method. Each cycle was performed at either 37° C. or 25° C. at a flow rate of 10 μL/min for antibody capture to the PrismA chip and 100 μL/min for analyte association (with 120 sec contact time) and dissociation (900 sec). Each cycle consisted of the following steps: injection of antibody at 1 μg/mL in HBS-EP+ targeting Rmax values of 60 RU on flow cell, injection of analyte in HBS-EP+ (concentration range of 500 nM to 0.25 nM by two-fold serial dilution for human LAG-3-His ECD (SEQ ID NO: 59) followed by 900-second dissociation phase, and regeneration using 10 μL of 10 mM glycine hydrochloride, pH 1.5 over a 60-second contact time utilizing a 10 μL/min flow rate. All analyte concentrations were determined utilizing monomeric molecular weight (MW) values. Association rates (kₒₙ) and dissociation rates (k_{off}) for LAG-3-ECD were evaluated using double referencing by flow-cell one reference subtraction in addition to 0 nM blank subtraction and fit to “1:1 (Langmuir) binding” model in the BIAevaluation software version 4.1. The dissociation constant (K_{D}) was calculated from the binding kinetics according to the relationship K_{D}=K_{off}/Kₒₙ. Stoichiometry=[RUₘₐₓ/RU_{captured}]/[MW_{analyte}/MW_{antibody}] where MW_{Antibody A} is 150 kDa. Values are reported as mean±standard deviation.

In experiments performed essentially as described above, the results in Table 2 were obtained. The results in Table 2 demonstrate that Antibody A, C, and D bind with high affinity to human and cynomolgus LAG-3-ECD.

TABLE 2

| | | | |
|---|---|---|---|
| | On Rate | Off Rate | Affinity |
| | (kₒₙ) | (k_{off}) | (K_{D}) |
| Species | (M⁻¹s⁻¹) | (s⁻¹) | (M) |

LAG-3 binding data for Antibody A

| | | | | |
|---|---|---|---|---|
| 37° C. | Human LAG-3-ECD | 2.1E+7 | 1.3E−3 | 6.4E−11 |
| | Cynomolgus LAG-3-ECD | 2.7E+6 | 3.5E−4 | 1.3E−10 |
| 25° C. | Human LAG-3-ECD | 8.3E+6 | 1.2E−3 | 1.4E−10 |
| | Cynomolgus LAG-3-ECD | 3.9E+6 | 1.5E−4 | 3.8E−11 |

LAG-3 binding data for Antibody C

| | | | | |
|---|---|---|---|---|
| 25° C. | Human LAG-3-ECD | 9.7E+4 | 8.9E−4 | 9.2E−9 |
| | Cynomolgus LAG-3-ECD | 1.2E+6 | 1.1E−3 | 9.4E−10 |

LAG-3 binding data for Antibody D

| | | | | |
|---|---|---|---|---|
| 25° C. | Human LAG-3-ECD | 2.7E+5 | 1.1E−4 | 4.1E−10 |
| | Cynomolgus LAG-3-ECD | 1.0E+6 | 1.9E−3 | 1.9E−9 |

### Peripheral Blood Mononuclear Cell Binding Assay

The ability of Antibody A to bind to cell surface human or cynomolgus monkey LAG-3 can be measured using a flow cytometry assay.

Briefly, human peripheral blood mononuclear cells (PBMCs) were isolated from healthy human Trima LRS (San Diego Blood Bank; San Diego, CA) using Ficoll (GE Healthcare #17144002), and activated with 4 ng/ml Staphylococcal Enterotoxin B (SEB) (Toxin Technologies, BT2021 MG) for 3 days in Complete RPMI (RPMI, Corning #MT10041CV; 10% FBS, Corning #35-011-CV; 1× Glutamax, Gibco #35050061; 1×P/S, Corning #30002CI; 1×BME, Gibco #21985023; 1×MEM, Gibco #11140050, 1× Sodium Pyruvate, Corning #25-000-CI) at 37° C. 5% CO2.

Cynomolgus PBMCs were isolated from whole blood (BioIVT #NHP01WBK2-0000861) using 90% Ficoll in PBS (Corning #21-031-CM), and activated with 4 ng/ml Staphylococcal Enterotoxin B (SEB) (Toxin Technologies, BT2021 MG) for 3 days in the RPMI as described above.

Cells were incubated with a 12 point, 4-fold serial dilutions of Antibody A or Isotype hG1, starting at 100 μg/mL, in PBS+2% FBS for 30 minutes at 4° C. The samples were washed and stained with 1 μg/mL Alexa Fluor 647 AffiniPure F (ab′) 2 Fragment Goat Anti-Human IgG, Fcy fragment specific (Jackson Immuno Research 109-606-098) in PBS+2% FBS for 30 minutes at 4° C. After washing thoroughly human samples were stained with CD3 Biolegend #300424, CD4 Biolegend #300512, CD8 Biolegend #301006, PD-1 Biolegend 329906, non-competing LAG3 418611 conjugated in house BN20-2739-10, Live/Dead Near IR Invitrogen L34975A, CD19 Biolegend 302218, CD33 Biolegend 366614, and CD56 Biolegend 362512 in PBS+2% FBS for 30 minutes at 4° C. Cyno samples were stained with CD3 BD 557917, CD4 BD 566910, CD8 BD 557746, PD-1 Biolegend 329906, non-competing LAG3 418611 conjugated in house BN20-2739-10, and Live/Dead Near IR Invitrogen L34975A in PBS+2% FBS for 30 minutes at 4° C. Cells were washed and processed on a BioRad ZE5 cytometer and data was analyzed with FlowJo software (BD Biosciences). Percent of cells bound by the respective antibody is quantified and graphed with Prism (Graphpad, San Diego, CA).

In experiments performed essentially as described above, the data in Table 3 were obtained. Antibody A bound cell membrane-expressed human LAG-3 and cynomolgus LAG-3 in Table 3 (human n=5 cyno n=3).

TABLE 3
Peripheral Mononuclear Cell Binding by Antibody A

| | Human | | Cyno | |
|---|---|---|---|---|
| | CD4 | CD8 | CD4 | CD8 |
| | EC50 +/− | EC50 +/− | EC50 +/− | EC50 +/− |
| | SEM, | SEM, | SEM, | SEM, |
| Molecule | (ng/mL) | (ng/mL) | (ng/mL) | (ng/mL) |
| Antibody A | 15.59 | 15.49 | 857.37 | 182.54 |
| | ng/mL +/− | ng/mL +/− | ng/mL +/− | ng/mL +/− |
| | 1.56 ng/mL | 5.24 ng/mL | 526.25 ng/mL | 88.42 ng/mL |
| Isotype hG1 | 389.96 | 675.88 | 2445167 | 8658.67 |
| | ng/mL +/− | ng/mL +/− | ng/mL +/− | ng/mL +/− |
| | 73.229 | 208.26 | 2428919 | 1575.83 |
| | ng/mL | ng/mL | ng/mL | ng/mL |

### In Vitro Inhibition of T-Cell Proliferation

The ability of the anti-human LAG-3 agonist antibodies of the invention to inhibit T cell proliferation was measured as follows:

Briefly, human peripheral blood mononuclear cells (PBMCs) were isolated from healthy human Trima LRS (San Diego Blood Bank; San Diego, CA) using Ficoll (GE Healthcare #17144002). Isolated PBMCs are labeled with a proliferation dye (Invitrogen Cat #6084290) for 20 minutes at 37° C., then washed thoroughly.

Labeled PBMCs were treated with a 12 point, 4-fold serial dilution of the respective antibodies starting at 100 μg/mL final in Complete RPMI (RPMI, Corning #MT10041CV; 10% FCS, Corning #MT35011CV; 1× Glutamax, Gibco #35050061; 1×P/S, Corning #30002CI; 1×BME, Gibco #21985023; 1×MEM, Gibco #11140050, 1× Sodium Pyruvate, Corning #25-000-CI) for 30 minutes at room temp. Cells were then stimulated with 4 ng/ml SEB (Toxin Technologies, BT2021 MG) final in Complete RPMI for 3 days at 37° C. with 5% CO2. Cells were then washed thoroughly.

Samples were stained with CD3 Biolegend #300424, CD4 Biolegend #300512, CD8 Biolegend #301006, PD-1 Biolegend 329906, non-competing LAG3 418611 conjugated in house BN20-2739-10, Live/Dead Near IR Invitrogen L34975A, CD19 Biolegend 302218, CD33 Biolegend 366614, and CD56 Biolegend 362512 in PBS+2% FBS for 30 minutes at 4° C. After washing thoroughly, cells were processed on a BioRad ZE5 cytometer, and data was analyzed with Flowjo software (BD Biosciences). Percent of cells proliferated was quantified and graphed with Prism (Graphpad, San Diego, CA).

In experiments performed essentially as described above, Antibody A inhibited human primary T cell proliferation, as shown in Table 4 (n=9).

TABLE 4
Inhibition of Human T cell Proliferation by Antibody A

| | CD4 IC₅₀ +/− | CD8 IC₅₀ +/− |
|---|---|---|
| Molecule | SEM (ng/mL) | SEM (ng/mL) |
| Antibody A | 135.99 ng/mL +/− | 45.70 ng/mL +/− |
| | 33.63 ng/mL | 6.54 ng/mL |
| Isotype hG1 | 3641.47 ng/mL +/− | 12470.75 ng/mL +/− |
| | 1866.79 ng/mL | 10385.22 ng/mL |

### In Vivo Inhibition of Graft-Versus-Host Disease (GVHD) in a Humanized Mouse Model

To demonstrate the immune modulatory activity of a LAG-3 agonist antibody of the invention, a humanized model of xenogeneic GvHD can be utilized. The model is generated by the engraftment of immunodeficient mice with human PBMCs. Human immune cells recognize the mouse as foreign and mount an immune response resulting in significant increases in human pro-inflammatory cytokines, immune cell activation, expansion and extravasation into tissues, ultimately resulting in weight loss and multi-system organ failure. Importantly, the inflammatory response is driven by human cells and thus human specific treatments can be interrogated in the model.

Briefly described, female NSG mice (NOD.Cg-Prkdcscid Il2rgtm1Wjl/SzJ, JAX Labs, Stock #05557) are housed 3 per cage at 72° C. under a 12 hour light: dark cycle and allowed food and water ad libitum (n=76). Human PBMCs are isolated from an LRS tubes obtained from blood donor (San Diego Blood Bank) using SepMate 50 Ficoll preparation tubes according to the manufacturer's instructions (STEMCELL Technologies, Vancouver, BC). Freshly isolated PBMCs are suspended in PBS at 1.2×10⁸ cells/mL and mice are engrafted with 100 μL PBMC suspension intravenously on day 0 (1.2×10⁷/mouse); 72 mice receive PBMCs and 4 mice remain non-engrafted controls.

On day 1 post engraftment, mice are divided into 9 weight matched (n-groups (n=8/group) and dosed subcutaneously with the antibody at 0.1, 1.0, or 3.0 mg/kg. Dosing continues weekly for the remainder of the experiment. Health checks and body weight measurements are performed routinely. Mice that lose 20% of their starting weight or are in obvious distress are euthanized. Clinical signs common to this model are scruffy hair, hunched body, wasting, and labored breathing or movement.

When the majority of isotype control mice are in need of euthanasia due to the progression of disease, all mice are sacrificed. For all sacrificed mice, blood is collected by cardiac puncture under isoflurane anesthesia into EDTA tubes; additionally, an interim blood sample on Day 10 is obtained by retro-orbital sinus. Blood from both collections is clarified by centrifugation for human plasma cytokine analysis. Body weight change is calculated as a percentage of their baseline weight: (Day (x) weight/Day 0 weight)*100. Mice requiring euthanasia prior to the end of the study have the last body weight measurement permutated to the end. Plasma cytokines are measured using the Mesoscale Discovery Human Th1/Th2 10-Vplex (Rockville, Maryland) according to the manufacturer's instructions. Data are graphed and statistics are calculated using Prism Software (GraphPad, San Diego, CA). Differences in weights between groups are determined by 2-way RM-ANOVA with Tukey's post hoc test. Differences in plasma cytokine levels are determined by 1-way ANOVA with Tukey's post hoc test. Differences between test groups are considered significant if p<0.05.

Engraftment of human PBMCs from elicited GvHD, as evidenced by marked wasting in NSG mice, which required study termination on Day 36 post engraftment. Treatments with Antibody A can significantly attenuate disease progression, as measured by a reduction in weight loss in mice in a dose dependent manner. Additionally, a human LAG-3 agonist antibody can inhibit the pronounced increase in plasma human pro-inflammatory cytokines associated with disease progression. Therefore, the anti-human LAG-3 agonist antibodies of the invention attenuate the human immune cell pathogenicity, and can reduce disease progression in a humanized GvHD model.

### Epitope Mapping by Hydrogen Deuterium Exchange Mass Spectrometry (HDX-MS)

Hydrogen deuterium exchange coupled with mass spectrometry (HDX-MS) was performed to determine where Antibody A binds the LAG-3 extracellular domain (LAG-3-ECD (e.g., SEQ ID NO: 59).

Peptide identification for LAG-3-ECD was performed on a Waters Synapt G2Si Mass Spec. (Waters Corporation™) instrument using 5 μg of LAG-3-ECD protein at zero exchange (1:10 dilution in 0.1× phosphate buffered saline in H₂O) using nepenthesin II (Nep II) for digestion. The mass spectrometer was set in HDMSe (Mobility ESI+ mode) using a mass acquisition range of m/z 255.00-1950.00 with a scan time of 0.4 s. Data was processed using ProteinLynx Global SERVER™ (PLGS) 2.3.02 (Waters Corporation™). For the exchange experiments, the complex of LAG-3-ECD protein with individual anti-human LAG-3 binding protein was prepared at the molar ratio of 1:1.2 in 10 mM sodium phosphate buffer, pH 7.4 containing 150 mM NaCl (1×PBS buffer). The experiment was initiated by adding 25 μL of D20 buffer containing 0.1×PBS to 2.5 μL of LAG-3-ECD (0.9 mg/mL) or LAG-3-ECD+protein complex at 15° C. for various amounts of time (0 s, 10 s, 2 min, 10 min and 60 min) using a custom TECAN sample preparation system (Espada et al., J Am Soc Mass Spectrom. (12): 2580-2583 (2019), which is hereby incorporated by reference in its entirety). The reaction was quenched using equal volume of was 0.32M TCEP, 3 M guanidine HCl, 0.1M phosphate pH 2.5 for two minutes at 4° C. and immediately frozen at −70° C. The sample injection system was comprised of a UR3 robot, a LEAP PAL3 HDX autosampler, and a high-performance liquid chromatography (HPLC) system interfaced with a Waters Synapt G2Si Mass Spec. (Waters Corporation™), with modification as described (Espada et al., 2019, J Am Soc Mass Spectrom. (12): 2580-2583 (2019), which is hereby incorporated by reference in its entirety). The LC mobile phases consisted of water (A) and acetonitrile (B), each containing 0.2% formic acid. Each sample was thawed using 50 μL of 1.5 M guanidine HCl, 0.1M phosphate pH 2.5, for 1 min and injected on to a Nep II column for digestion at 4° C. with mobile phase A at a flow rate of 250 μL/min for 2.5 minutes. The resulting peptides were trapped on a Waters BEH Vanguard Pre-column at 4° C., and chromatographically separated using a Waters Acquity UPLC BEH C18 analytical column at 4° C. with a flow rate of 200 μL/min and a gradient of 3%-85% mobile phase B over 7 minutes and directed into mass spectrometer for mass analysis. The Synapt G2Si was calibrated with Glu-fibrinopeptide (Waters Corporation™) prior to use. Mass spectra were acquired over the m/z range of 255 to 1950 in HDMS mode, with the lock mass m/z of 556.2771 (Leucine Enkephalin, Waters Corporation™). The relative deuterium incorporation for each peptide was determined by processing the MS data for deuterated samples along with the undeuterated control using the identified peptide list in DynamX 3.0 (Waters Corporation™). The free and bound states of LAG-3-ECD were compared for deuterium incorporation differences to identify protected regions indicative of the binding epitope.

For Antibody A, a substantial decrease in deuterium uptake upon binding to LAG-3-ECD was observed in residues PDRPASVHWFRNRGQGRVPVRESPHHHLAESF (SEQ ID NO: 62) and PQVSPMD (SEQ ID NO: 63), indicating the epitope region for the Antibody 1. A decrease in deuterium uptake upon binding to LAG-3-ECD was also observed in residues SPHHHLAESF (SEQ ID NO: 64). Taken together the HDX-MS data indicate the anti-human LAG-3 agonist antibodies of the invention bind largely, if not entirely within Domain 2 of LAG-3.

### Listing of Amino Acid and Nucleotide Sequences

Antibody A
HCDR1:
TVSGGSISSGGYYWS (SEQ ID NO: 1)
HCDR2:
YIYYSGITYYNPSLKS (SEQ ID NO: 2)
HCDR3:
ARSAVTTPFDY (SEQ ID NO: 3)
LCDR1:
KSSQSILYSSDNKNYLA (SEQ ID NO: 4)
LCDR2:
YWASTRES (SEQ ID NO: 5)
LCDR3:
QQYYSPPWT (SEQ ID NO: 6)
VH:
QVQLQESGPGLVKPSQTLSLTCTVSGGSISSGGYYWSWIRQHPGKGLEWIGYIYYSGITY
YNPSLKSRVTISVDTSKNQFSLKLSSVTAADTAVYYCARSAVTTPFDYWGQGTLVTVSS
(SEQ ID NO: 7)
VL:
DIVMTQSPDSLAVSLGERATINCKSSQSILYSSDNKNYLAWYQQKPGQPPKLLIYWASTR
ESGVPDRFSGSGSGTDFTLTISSLQAEDVAVYYCQQYYSPPWTFGQGTKVEIK
(SEQ ID NO: 8)
Constant region of heavy chain:
ASTKGPSVFPLAPSSKSTSGGTAALGCLVKDYFPEPVTVSWNSGALTSGVHTFPAVLQSS
GLYSLSSVVTVPSSSLGTQTYICNVNHKPSNTKVDKRVEPKSCDKTHTCPPCPAPELLGG
PSVFLFPPKPKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREPQVYTLPPSR
EEMTKNQVSLTCLVKGFYPSDIAVEWESNGQPENNYKTTPPVLDSDGSFFLYSKLTVDK
SRWQQGNVFSCSVMHEALHNHYTQKSLSLSPGK (SEQ ID NO: 9)
Constant region of light chain:
RTVAAPSVFIFPPSDEQLKSGTASVVCLLNNFYPREAKVQWKVDNALQSGNSQESVTEQ
DSKDSTYSLSSTLTLSKADYEKHKVYACEVTHQGLSSPVTKSFNRGEC (SEQ ID NO: 10)
Heavy chain:
QVQLQESGPGLVKPSQTLSLTCTVSGGSISSGGYYWSWIRQHPGKGLEWIGYIYYSGITY
YNPSLKSRVTISVDTSKNQFSLKLSSVTAADTAVYYCARSAVTTPFDYWGQGTLVTVSS
ASTKGPSVFPLAPSSKSTSGGTAALGCLVKDYFPEPVTVSWNSGALTSGVHTFPAVLQSS
GLYSLSSVVTVPSSSLGTQTYICNVNHKPSNTKVDKRVEPKSCDKTHTCPPCPAPELLGG
PSVFLFPPKPKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREPQVYTLPPSR
EEMTKNQVSLTCLVKGFYPSDIAVEWESNGQPENNYKTTPPVLDSDGSFFLYSKLTVDK
SRWQQGNVFSCSVMHEALHNHYTQKSLSLSPGK (SEQ ID NO: 11)
Light chain:
DIVMTQSPDSLAVSLGERATINCKSSQSILYSSDNKNYLAWYQQKPGQPPKLLIYWASTR
ESGVPDRFSGSGSGTDFTLTISSLQAEDVAVYYCQQYYSPPWTFGQGTKVEIKRTVAAPS
VFIFPPSDEQLKSGTASVVCLLNNFYPREAKVQWKVDNALQSGNSQESVTEQDSKDSTY
SLSSTLTLSKADYEKHKVYACEVTHQGLSSPVTKSFNRGEC (SEQ ID NO: 12)
DNA encoding heavy chain:
CAGGTGCAGCTGCAGGAGTCGGGCCCAGGACTGGTGAAGCCTTCACAGACCCTGTC
CCTCACCTGCACTGTCTCTGGTGGCTCCATCAGCAGTGGTGGTTACTACTGGAGCTG
GATCCGCCAGCACCCAGGGAAGGGCCTGGAGTGGATTGGGTACATCTATTACAGTG
GGATAACCTACTACAACCCGTCCCTCAAGAGTCGAGTTACCATATCAGTAGACACGT
CTAAGAACCAGTTCTCCCTGAAGCTGAGCTCTGTGACTGCCGCGGACACGGCCGTGT
ATTACTGTGCGAGATCAGCAGTGACCACCCCCTTCGACTACTGGGGACAGGGAACTC
TGGTGACGGTGTCCTCCGCCAGCACCAAGGGCCCATCGGTCTTCCCCCTGGCACCCT
CCTCCAAGAGCACCTCTGGGGGCACAGCGGCCCTGGGCTGCCTGGTCAAGGACTACT
TCCCCGAACCGGTGACGGTGTCGTGGAACTCAGGCGCACTGACCAGCGGCGTGCAC
ACCTTCCCGGCTGTCCTACAGTCCTCAGGACTCTACTCCCTCAGCAGCGTGGTGACC
GTGCCCTCCAGCAGCTTGGGCACCCAGACCTACATCTGCAACGTGAATCACAAGCCC
AGCAACACCAAGGTGGACAAGAGAGTTGAGCCCAAATCTTGTGACAAAACTCACAC
ATGCCCACCGTGCCCAGCACCTGAACTCCTGGGGGGACCGTCAGTCTTCCTCTTCCC
CCCAAAACCCAAGGACACCCTCATGATCTCCCGGACCCCTGAGGTCACATGCGTGGT
GGTGGACGTGAGCCACGAAGACCCTGAGGTCAAGTTCAACTGGTATGTGGACGGCG
TGGAGGTGCATAATGCCAAGACAAAGCCGCGGGAGGAGCAGTACAACAGCACGTAC
CGTGTGGTCAGCGTCCTCACCGTCCTGCACCAAGACTGGCTGAATGGCAAGGAGTAC
AAGTGCAAGGTCTCCAACAAAGCCCTCCCAGCCCCCATCGAGAAAACCATCTCCAA
AGCCAAAGGGCAGCCCCGAGAACCACAGGTGTACACCCTGCCCCCATCCCGGGAGG
AGATGACCAAGAACCAAGTCAGCCTGACCTGCCTGGTCAAAGGCTTCTATCCCAGCG
ACATCGCCGTGGAGTGGGAGAGCAATGGGCAGCCGGAGAACAACTACAAGACCACG
CCTCCCGTGCTGGACTCCGACGGCTCCTTCTTCCTCTATTCCAAGCTCACCGTGGACA
AGAGCAGGTGGCAGCAGGGGAACGTCTTCTCATGCTCCGTGATGCATGAGGCTCTGC
ACAACCACTACACGCAGAAGAGCCTCTCCCTGTCTCCGGGCAAA (SEQ ID NO: 13)
DNA encoding light chain:
GACATCGTGATGACCCAGTCTCCAGACTCCCTGGCTGTGTCTCTGGGCGAGAGGGCC
ACCATCAACTGCAAGTCCAGCCAGAGTATATTATACAGCTCCGACAATAAGAACTAC
TTAGCTTGGTACCAGCAGAAACCAGGACAGCCTCCTAAGCTGCTCATTTACTGGGCA
TCTACCCGGGAATCCGGGGTCCCTGACCGATTCAGTGGCAGCGGGTCTGGGACAGAT
TTCACTCTCACCATCAGCAGCCTGCAGGCTGAAGATGTGGCAGTTTATTACTGTCAG
CAGTACTACTCACCACCATGGACGTTCGGGCAGGGAACTAAGGTGGAAATAAAGCG
AACCGTGGCTGCACCATCTGTCTTCATCTTCCCGCCATCTGATGAGCAGTTGAAATCT
GGAACTGCCTCTGTTGTGTGCCTGCTGAATAACTTCTATCCCAGAGAGGCCAAAGTA
CAGTGGAAGGTGGATAACGCCCTCCAATCGGGTAACTCCCAGGAGAGTGTCACAGA
GCAGGACAGCAAGGACAGCACCTACAGCCTCAGCAGCACCCTGACGCTGAGCAAAG
CAGACTACGAGAAACACAAAGTCTACGCCTGCGAAGTCACCCATCAGGGCCTGAGC
TCGCCCGTCACAAAGAGCTTCAACAGGGGAGAGTGC (SEQ ID NO: 14)
Antibody B
HCDR1:
TVSGGSISSGGYYWS (SEQ ID NO: 1)
HCDR2:
YIYYSGITYYNPSLKS (SEQ ID NO: 2)
HCDR3:
ASSTVTTPFDY (SEQ ID NO: 15)
LCDR1:
KSSQSVLYSSNNKNYLA (SEQ ID NO: 16)
LCDR2:
YWASTRES (SEQ ID NO: 5)
LCDR3:
QQYYSPPWT (SEQ ID NO: 6)
VH:
X₁VQLQESGPGLVKPSQTLSLTCTVSGGSISSGGYYWSWIRQHPGKGLEWIGYIYYSGITY
YNPSLKSRVTISVDTSKNQFSLKLSSVTAADTAVYYCASSTVTTPFDYWGQGTLVTVSS
(SEQ ID NO: 17)
wherein X₁ is Q, pyroglutamate, or absent
VL:
DIVMTQSPDSLAVSLGERATINCKSSQSVLYSSNNKNYLAWYQQKPGQPPKLLIYWAST
RESGVPDRFSGSGSGTDFTLTISSLQAEDVAVYYCQQYYSPPWTFGQGTKVEIK (SEQ ID
NO: 18)
Heavy chain:
X₁VQLQESGPGLVKPSQTLSLTCTVSGGSISSGGYYWSWIRQHPGKGLEWIGYIYYSGITY
YNPSLKSRVTISVDTSKNQFSLKLSSVTAADTAVYYCASSTVTTPFDYWGQGTLVTVSSA
STKGPSVFPLAPSSKSTSGGTAALGCLVKDYFPEPVTVSWNSGALTSGVHTFPAVLQSSG
LYSLSSVVTVPSSSLGTQTYICNVNHKPSNTKVDKRVEPKSCDKTHTCPPCPAPELLGGPS
VFLFPPKPKDTLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQVYTLPPSREE
MTKNQVSLTCLVKGFYPSDIAVEWESNGQPENNYKTTPPVLDSDGSFFLYSKLTVDKSR
WQQGNVFSCSVMHEALHNHYTQKSLSLSPGX₂ (SEQ ID NO: 19)
wherein X₁ is Q, pyroglutamate, or absent and X₂ is K or absent
Light chain:
DIVMTQSPDSLAVSLGERATINCKSSQSVLYSSNNKNYLAWYQQKPGQPPKLLIYWAST
RESGVPDRFSGSGSGTDFTLTISSLQAEDVAVYYCQQYYSPPWTFGQGTKVEIKRTVAAP
SVFIFPPSDEQLKSGTASVVCLLNNFYPREAKVQWKVDNALQSGNSQESVTEQDSKDST
YSLSSTLTLSKADYEKHKVYACEVTHQGLSSPVTKSFNRGEC (SEQ ID NO: 20)
Antibody C
HCDR1:
TVSGVSITSYYWS (SEQ ID NO: 21)
HCDR2:
RIFTSGSTNYNPSLKS (SEQ ID NO: 22)
HCDR3:
ARYDGME (SEQ ID NO: 23)
LCDR1:
RASQSVSSSYLA (SEQ ID NO: 24)
LCDR2:
YGASSRAT (SEQ ID NO: 25)
LCDR3:
QQYGSSPLT (SEQ ID NO: 26)
VH:
X₁VQLQESGPGLVKPSETLSLTCTVSGVSITSYYWSWIRQPAGKGLEWIGRIFTSGSTNYN
PSLKSRVTMSVDTSKNQFSLKLSSVTAADTAVYYCARYDGMEWGQGTLVTVSS (SEQ
ID NO: 27)
wherein X₁ is Q, pyroglutamate, or absent
VL:
X₁IVLTQSPGTLSLSPGERATLSCRASQSVSSSYLAWYQQKPGQAPRLLIYGASSRATGIP
DRFSGSGSGTDFTLTISRLEPEDFAVYYCQQYGSSPLTFGQGTKVEIK (SEQ ID NO: 28)
wherein X₁ is E, pyroglutamate, or absent
Heavy chain:
X₁VQLQESGPGLVKPSETLSLTCTVSGVSITSYYWSWIRQPAGKGLEWIGRIFTSGSTNYN
PSLKSRVTMSVDTSKNQFSLKLSSVTAADTAVYYCARYDGMEWGQGTLVTVSSASTKG
PSVFPLAPSSKSTSGGTAALGCLVKDYFPEPVTVSWNSGALTSGVHTFPAVLQSSGLYSL
SSVVTVPSSSLGTQTYICNVNHKPSNTKVDKRVEPKSCDKTHTCPPCPAPELLGGPSVFLF
PPKPKDTLMISRTPEVTCVVVDVSHEDPEVKFNWYVDGVEVHNAKTKPREEQYNSTYR
VVSVLTVLHQDWLNGKEYKCKVSNKALPAPIEKTISKAKGQPREPQVYTLPPSREEMTK
NQVSLTCLVKGFYPSDIAVEWESNGQPENNYKTTPPVLDSDGSFFLYSKLTVDKSRWQQ
GNVFSCSVMHEALHNHYTQKSLSLSPGX₂ (SEQ ID NO: 29)
wherein X₁ is Q, pyroglutamate, or absent and X₂ is K or absent
Light chain:
X₁IVLTQSPGTLSLSPGERATLSCRASQSVSSSYLAWYQQKPGQAPRLLIYGASSRATGIP
DRFSGSGSGTDFTLTISRLEPEDFAVYYCQQYGSSPLTFGQGTKVEIKRTVAAPSVFIFPPS
DEQLKSGTASVVCLLNNFYPREAKVQWKVDNALQSGNSQESVTEQDSKDSTYSLSSTLT
LSKADYEKHKVYACEVTHQGLSSPVTKSFNRGEC (SEQ ID NO: 30)
wherein X₁ is E, pyroglutamate, or absent
Antibody D
HCDR1:
TVSGGSIRSYYWS (SEQ ID NO: 31)
HCDR2:
RIFTSGSTNYNPSLKS (SEQ ID NO: 22)
HCDR3:
ARYEGFDV (SEQ ID NO: 32)
LCDR1:
RASQSVSSSYLA (SEQ ID NO: 24)
LCDR2:
YGASSRAT (SEQ ID NO: 25)
LCDR3:
QQYGSSPIT (SEQ ID NO: 33)
VH:
X₁VQLQESGPGLVKPSETLSLTCTVSGGSIRSYYWSWIRQPAGKGLEWIGRIFTSGSTNYN
PSLKSRVTMSVDTSKNQFSLKLSSVTAADTAVYYCARYEGFDVWGQGTLVTVSS (SEQ
ID NO: 34)
wherein X₁ is Q, pyroglutamate, or absent
VL:
X₁IVLTQSPGTLSLSPGERATLSCRASQSVSSSYLAWYQQKPGQAPRLLIYGASSRATGIP
DRFSGSGSGTDFTLTISRLEPEDFAVYYCQQYGSSPITFGQGTKVEIK (SEQ ID NO: 35)
wherein X₁ is E, pyroglutamate, or absent
Heavy chain:
X₁VQLQESGPGLVKPSETLSLTCTVSGGSIRSYYWSWIRQPAGKGLEWIGRIFTSGSTNYN
PSLKSRVTMSVDTSKNQFSLKLSSVTAADTAVYYCARYEGFDVWGQGTLVTVSSASTK
GPSVFPLAPSSKSTSGGTAALGCLVKDYFPEPVTVSWNSGALTSGVHTFPAVLQSSGLYS
LSSVVTVPSSSLGTQTYICNVNHKPSNTKVDKRVEPKSCDKTHTCPPCPAPELLGGPSVFL
FPPKPKDTLMISRTPEVTCVVVDVSHEDPEVKFNWYVDGVEVHNAKTKPREEQYNSTY
RVVSVLTVLHQDWLNGKEYKCKVSNKALPAPIEKTISKAKGQPREPQVYTLPPSREEMT
KNQVSLTCLVKGFYPSDIAVEWESNGQPENNYKTTPPVLDSDGSFFLYSKLTVDKSRWQ
QGNVFSCSVMHEALHNHYTQKSLSLSPGX₂ (SEQ ID NO: 36)
wherein X₁ is Q, pyroglutamate, or absent and X₂ is K or absent
Light chain:
X₁IVLTQSPGTLSLSPGERATLSCRASQSVSSSYLAWYQQKPGQAPRLLIYGASSRATGIP
DRFSGSGSGTDFTLTISRLEPEDFAVYYCQQYGSSPITFGQGTKVEIKRTVAAPSVFIFPPS
DEQLKSGTASVVCLLNNFYPREAKVQWKVDNALQSGNSQESVTEQDSKDSTYSLSSTLT
LSKADYEKHKVYACEVTHQGLSSPVTKSFNRGEC (SEQ ID NO: 37)
wherein X₁ is E, pyroglutamate, or absent
Antibody E
HCDR1:
AASGFTFSSFAMS (SEQ ID NO: 38)
HCDR2:
AISASGGRTYYADSVKG (SEQ ID NO: 39)
HCDR3:
AKDEPYSGSYHGLDV (SEQ ID NO: 40)
LCDR1:
RSSQSLLHSDGYNYLD (SEQ ID NO: 41)
LCDR2:
YLGSNRAS (SEQ ID NO: 42)
LCDR3:
MQAQQTPLT (SEQ ID NO: 43)
VH:
X₁VQLVESGGGLVQPGGSLRLSCAASGFTFSSFAMSWVRQAPGKGLEWVSAISASGGRT
YYADSVKGRFTISRDNSKNTLYLQMNSLRAEDTAVYYCAKDEPYSGSYHGLDVWGQG
TLVTVSS (SEQ ID NO: 44)
wherein X₁ is Q, pyroglutamate, or absent
VL:
DIVMTQSPLSLPVTPGEPASISCRSSQSLLHSDGYNYLDWYLQKPGQSPQLLIYLGSNRAS
GVPDRFSGSGSGTDFTLKISRVEAEDVGVYYCMQAQQTPLTFGQGTKVEIK (SEQ ID
NO: 45)
Heavy chain:
X₁VQLVESGGGLVQPGGSLRLSCAASGFTFSSFAMSWVRQAPGKGLEWVSAISASGGRT
YYADSVKGRFTISRDNSKNTLYLQMNSLRAEDTAVYYCAKDEPYSGSYHGLDVWGQG
TLVTVSSASTKGPSVFPLAPSSKSTSGGTAALGCLVKDYFPEPVTVSWNSGALTSGVHTF
PAVLQSSGLYSLSSVVTVPSSSLGTQTYICNVNHKPSNTKVDKRVEPKSCDKTHTCPPCP
APELLGGPSVFLFPPKPKDTLMISRTPEVTCVVVDVSHEDPEVKFNWYVDGVEVHNAKT
KPREEQYNSTYRVVSVLTVLHQDWLNGKEYKCKVSNKALPAPIEKTISKAKGQPREPQV
YTLPPSREEMTKNQVSLTCLVKGFYPSDIAVEWESNGQPENNYKTTPPVLDSDGSFFLYS
KLTVDKSRWQQGNVFSCSVMHEALHNHYTQKSLSLSPGX₂ (SEQ ID NO: 46)
wherein X₁ is Q, pyroglutamate, or absent and X₂ is K or absent
Light chain:
DIVMTQSPLSLPVTPGEPASISCRSSQSLLHSDGYNYLDWYLQKPGQSPQLLIYLGSNRAS
GVPDRFSGSGSGTDFTLKISRVEAEDVGVYYCMQAQQTPLTFGQGTKVEIKRTVAAPSV
FIFPPSDEQLKSGTASVVCLLNNFYPREAKVQWKVDNALQSGNSQESVTEQDSKDSTYS
LSSTLTLSKADYEKHKVYACEVTHQGLSSPVTKSFNRGEC (SEQ ID NO: 47)
Consensus sequences:
Group I: Antibody A and Antibody B
Consensus HCDR3:
AX₁SX₂VTTPFDY (SEQ ID NO: 48)
wherein X₁ is R or S and X₂ is A or T
Consensus LCDR1:
KSSQSX₁LYSSX₂NKNYLA (SEQ ID NO: 49)
wherein X₁ is I or V and X₂ is D or N
Consensus VH:
X₁VQLQESGPGLVKPSQTLSLTCTVSGGSISSGGYYWSWIRQHPGKGLEWIGYIYYSGITY
YNPSLKSRVTISVDTSKNQFSLKLSSVTAADTAVYYCAX₂SX₃VTTPFDYWGQGTLVTVS
S (SEQ ID NO: 50)
wherein X₁ is Q, pyroglutamate, or absent, X₂ is R or S and X₃ is A or T
Consensus LCVR:
DIVMTQSPDSLAVSLGERATINCKSSQSX₁LYSSX₂NKNYLAWYQQKPGQPPKLLIYWAS
TRESGVPDRFSGSGSGTDFTLTISSLQAEDVAVYYCQQYYSPPWTFGQGTKVEIK (SEQ
ID NO: 51)
wherein X₁ is I or V and X₂ is D or N
Group II: Antibody C and Antibody D
HCDR1:
TVSGX₁SIX₂SYYWS (SEQ ID NO: 52)
wherein X₁ is G or V and X₂ is R or T
HCDR3:
ARYX₁GX₂X₃X₄ (SEQ ID NO: 53)
wherein X₁ is E or D; X₂ is F or M; X₃ is D or E; X₄ is V or absent.
LCDR3:
QQYGSSPX₁T (SEQ ID NO: 54)
wherein X₁ is I or L.
VH:
X₁VQLQESGPGLVKPSETLSLTCTVSGX₂SIX₃SYYWSWIRQPAGKGLEWIGRIFTSGSTNY
NPSLKSRVTMSVDTSKNQFSLKLSSVTAADTAVYYCARYX₄GX₅X₆X₇WGQGTLVTVSS
(SEQ ID NO: 55)
wherein X₁ is Q, pyroglutamate, or absent; X₂ is G or V; X₃ is R or T; X₄ is E or D; X₅
is F or M; X₆ is D or E; X₇ is V or absent.
VL:
X₁IVLTQSPGTLSLSPGERATLSCRASQSVSSSYLAWYQQKPGQAPRLLIYGASSRATGIP
DRFSGSGSGTDFTLTISRLEPEDFAVYYCQQYGSSPX₂TFGQGTKVEIK (SEQ ID NO: 56)
wherein X₁ is E, pyroglutamate, or absent and X₂ is I or L.
Human LAG-3 amino acid sequence with signal sequence:
MWEAQFLGLLFLQPLWVAPVKPLQPGAEVPVVWAQEGAPAQLPCSPTIPLQDLSLLRRA
GVTWQHQPDSGPPAAAPGHPLAPGPHPAAPSSWGPRPRRYTVLSVGPGGLRSGRLPLQP
RVQLDERGRQRGDFSLWLRPARRADAGEYRAAVHLRDRALSCRLRLRLGQASMTASPP
GSLRASDWVILNCSFSRPDRPASVHWFRNRGQGRVPVRESPHHHLAESFLFLPQVSPMD
SGPWGCILTYRDGFNVSIMYNLTVLGLEPPTPLTVYAGAGSRVGLPCRLPAGVGTRSFLT
AKWTPPGGGPDLLVTGDNGDFTLRLEDVSQAQAGTYTCHIHLQEQQLNATVTLAIITVT
PKSFGSPGSLGKLLCEVTPVSGQERFVWSSLDTPSQRSFSGPWLEAQEAQLLSQPWQCQL
YQGERLLGAAVYFTELSSPGAQRSGRAPGALPAGHLLLFLILGVLSLLLLVTGAFGFHLW
RRQWRPRRFSALEQGIHPPQAQSKIEELEQEPEPEPEPEPEPEPEPEPEQL (SEQ ID NO: 57)
Human LAG-3 amino acid sequence without signal sequence:
LQPGAEVPVVWAQEGAPAQLPCSPTIPLQDLSLLRRAGVTWQHQPDSGPPAAAPGHPLA
PGPHPAAPSSWGPRPRRYTVLSVGPGGLRSGRLPLQPRVQLDERGRQRGDFSLWLRPAR
RADAGEYRAAVHLRDRALSCRLRLRLGQASMTASPPGSLRASDWVILNCSFSRPDRPAS
VHWFRNRGQGRVPVRESPHHHLAESFLFLPQVSPMDSGPWGCILTYRDGFNVSIMYNLT
VLGLEPPTPLTVYAGAGSRVGLPCRLPAGVGTRSFLTAKWTPPGGGPDLLVTGDNGDFT
LRLEDVSQAQAGTYTCHIHLQEQQLNATVTLAIITVTPKSFGSPGSLGKLLCEVTPVSGQE
RFVWSSLDTPSQRSFSGPWLEAQEAQLLSQPWQCQLYQGERLLGAAVYFTELSSPGAQR
SGRAPGALPAGHLLLFLILGVLSLLLLVTGAFGFHLWRRQWRPRRFSALEQGIHPPQAQS
KIEELEQEPEPEPEPEPEPEPEPEPEQL (SEQ ID NO: 58)
Human LAG-3 ECD-His:
LQPGAEVPVVWAQEGAPAQLPCSPTIPLQDLSLLRRAGVTWQHQPDSGPPAAAPGHPLA
PGPHPAAPSSWGPRPRRYTVLSVGPGGLRSGRLPLQPRVQLDERGRQRGDFSLWLRPAR
RADAGEYRAAVHLRDRALSCRLRLRLGQASMTASPPGSLRASDWVILNCSFSRPDRPAS
VHWFRNRGQGRVPVRESPHHHLAESFLFLPQVSPMDSGPWGCILTYRDGFNVSIMYNLT
VLGLEPPTPLTVYAGAGSRVGLPCRLPAGVGTRSFLTAKWTPPGGGPDLLVTGDNGDFT
LRLEDVSQAQAGTYTCHIHLQEQQLNATVTLAIITVTPKSFGSPGSLGKLLCEVTPVSGQE
RFVWSSLDTPSQRSFSGPWLEAQEAQLLSQPWQCQLYQGERLLGAAVYFTELSSPGAQR
SGRAPGALPAGHGGGGSHHHHHH (SEQ ID NO: 59)
Cynomolgus LAG-3 ECD-His:
PQPGAEISVVWAQEGAPAQLPCSPTIPLQDLSLLRRAGVTWQHQPDSGPPAPAPGHPPVP
GHRPAAPYSWGPRPRRYTVLSVGPGGLRSGRLPLQPRVQLDERGRQRGDFSLWLRPAR
RADAGEYRATVHLRDRALSCRLRLRVGQASMTASPPGSLRTSDWVILNCSFSRPDRPAS
VHWFRSRGQGRVPVQGSPHHHLAESFLFLPHVGPMDSGLWGCILTYRDGFNVSIMYNL
TVLGLEPATPLTVYAGAGSRVELPCRLPPAVGTQSFLTAKWAPPGGGPDLLVAGDNGDF
TLRLEDVSQAQAGTYICHIRLQGQQLNATVTLAIITVTPKSFGSPGSLGKLLCEVTPASGQ
EHFVWSPLNTPSQRSFSGPWLEAQEAQLLSQPWQCQLHQGERLLGAAVYFTELSSPGAQ
RSGRAPGALRAGHAAAHHHHHHSGS (SEQ ID NO: 60)
D2 of human LAG-3:
QASMTASPPGSLRASDWVILNCSFSRPDRPASVHWFRNRGQGRVPVRESPHHHLAESFL
FLPQVSPMDSGPWGCILTYRDGFNVSIMYNLTVLG (SEQ ID NO: 61)
Epitope Regions of D2:
PDRPASVHWFRNRGQGRVPVRESPHHHLAESF (SEQ ID NO: 62)
PQVSPMD (SEQ ID NO: 63)
SPHHHLAESF (SEQ ID NO: 64)
Antibody A total consensus
HCDR1:
TVSGGSISSGGYX₁WS (SEQ ID NO: 65)
wherein X₁ is F or Y
HCDR2:
(SEQ ID NO: 2)
HCDR3:
(SEQ ID NO: 48)
LCDR1:
KSSQSX₁LYSSX₂NX₃NYLA (SEQ ID NO: 66)
wherein X₁ is I or V; X₂ is D or N; X₃ is R or K
LCDR2:
YWASTRX₁S (SEQ ID NO: 67)
wherein X₁ is D or E
LCDR3:
X₁QYYSPPWT (SEQ ID NO: 68)
wherein X₁ is H or Q
VH:
X₁VQLQESGPGLVKPSQTLSLTCTVSGGSISSGGYX₂WSWIRQHPGKGLEWIGYIYYSGIT
YYNPSLKSRX₃TISVDTSKNQFSLX₄LSSVTAADTAVYYCAX₅SX₆VTTPFDYWGQGTLVT
VSS (SEQ ID NO: 69)
wherein X₁ is Q, pyroglutamate, or absent; X₂ is F or Y; X₃ is L or V; X₄ is N or K; X₅
is R or S; X₆ is A or T
VL:
DIVMTQSPDX₁LAVSLGERATINCKSSQSILYSSDNX₂NYLAWX₃QX₄KPGQ
PPKLLIYWASTRX₅SGVPDRFSGSGSGTDFTLX₆ISSLQAEDVAVYYCX₇QYY
SPPWTFGQGTKVEIK (SEQ ID NO: 70)
wherein X₁ is A or S, X₂ is R or K, X₃ is F or Y, X₄ is L or Q, X₅ is D or E, X₆ is A
or T, and X₇ is H or Q
HC:
X₁VQLQESGPGLVKPSQTLSLTCTVSGGSISSGGYX₂WSWIRQHPGKGLEWIGYIYYSGIT
YYNPSLKSRX₃TISVDTSKNQFSLX₄LSSVTAADTAVYYCARSAVTTPFDYWGQGTLVTV
SSASTKGPSVFPLAPSSKSTSGGTAALGCLVKDYFPEPVTVSWNSGALTSGVHTFPAVLQ
SSGLYSLSSVVTVPSSSLGTQTYICNVNHKPSNTKVDKRVEPKSCDKTHTCPPCPAPEX₅
X₆GGPSVFLFPPKPKDTLMISRTPEVTCVVVX₇VSHEDPEVKFNWYVDGVEVHNAKTKPR
EEQYNSTYRVVSVLTVLHQDWLNGKEYKCKVSNKALPAPIEKTISKAKGQPREPQVYTL
PPSREEMTKNQVSLTCLVKGFYPSDIAVEWESNGQPENNYKTTPPVLDSDGSFFLYSKLT
VDKSRWQQGNVFSCSVMHEALHNHYTQKSLSLSPGX₈ (SEQ ID NO: 71)
wherein X₁ is Q, pyroglutamate, or absent; X₂ is F or Y; X₃ is L or V; X₄ is N or K; X₅
is L or A; X₆ is L or A; X₇ is D or S; X₈ is K or absent
LC:
DIVMTQSPDX₁LAVSLGERATINCKSSQSX₂LYSSDNX₃NYLAWX₄QX₅KPGQPPKLLIYW
ASTRX₆SGVPDRFSGSGSGTDFTLX₇ISSLQAEDVAVYYCX₈QYYSPPWTFGQGTKVEIKR
TVAAPSVFIFPPSDEQLKSGTASVVCLLNNFYPREAKVQWKVDNALQSGNSQESVTEQD
SKDSTYSLSSTLTLSKADYEKHKVYACEVTHQGLSSPVTKSFNRGEC (SEQ ID NO: 72)
wherein X₁ is A or S, X₂ is I or V, X₃ is R or K, X₄ is F or Y, X₅ is L or Q, X₆ is D
or E, X₇ is A or T, and X₈ is H or Q
VH of Antibody A (N-terminal modification):
X₁VQLQESGPGLVKPSQTLSLTCTVSGGSISSGGYYWSWIRQHPGKGLEWIGYIYYSGITY
YNPSLKSRVTISVDTSKNQFSLKLSSVTAADTAVYYCARSAVTTPFDYWGQGTLVTVSS
(SEQ ID NO: 73)
wherein X₁ is Q, pyroglutamate, or absent
Constant region of heavy chain (C-terminal modification):
ASTKGPSVFPLAPSSKSTSGGTAALGCLVKDYFPEPVTVSWNSGALTSGVHTFPAVLQSS
GLYSLSSVVTVPSSSLGTQTYICNVNHKPSNTKVDKRVEPKSCDKTHTCPPCPAPELLGG
PSVFLFPPKPKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREPQVYTLPPSR
EEMTKNQVSLTCLVKGFYPSDIAVEWESNGQPENNYKTTPPVLDSDGSFFLYSKLTVDK
SRWQQGNVFSCSVMHEALHNHYTQKSLSLSPGX₁ (SEQ ID NO: 74)
wherein X₁ is K or absent
Heavy chain of Antibody A (with N- and C- terminal modifications):
X₁VQLQESGPGLVKPSQTLSLTCTVSGGSISSGGYYWSWIRQHPGKGLEWIGYIYYSGITY
YNPSLKSRVTISVDTSKNQFSLKLSSVTAADTAVYYCARSAVTTPFDYWGQGTLVTVSS
ASTKGPSVFPLAPSSKSTSGGTAALGCLVKDYFPEPVTVSWNSGALTSGVHTFPAVLQSS
GLYSLSSVVTVPSSSLGTQTYICNVNHKPSNTKVDKRVEPKSCDKTHTCPPCPAPELLGG
PSVFLFPPKPKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREPQVYTLPPSR
EEMTKNQVSLTCLVKGFYPSDIAVEWESNGQPENNYKTTPPVLDSDGSFFLYSKLTVDK
SRWQQGNVFSCSVMHEALHNHYTQKSLSLSPGX₂ (SEQ ID NO: 75)
wherein X₁ is Q, pyroglutamate, or absent and X₂ is K or absent

We claim:

1. An antibody that binds to Domain 2 of human LAG-3 (SEQ ID NO: 61), wherein said antibody comprises a heavy chain variable region (VH) and a light chain variable region (VL), wherein the VH comprises heavy chain complementarity determining regions (HCDR): HCDR1 comprising SEQ ID NO: 65, HCDR2 comprising SEQ ID NO: 2, and HCDR3 comprising SEQ ID NO: 48; and wherein the VL comprises light chain complementarity determining regions (LCDR): LCDR1 comprising SEQ ID NO: 66, LCDR2 comprising SEQ ID NO: 67, and LCDR3 comprising SEQ ID NO: 68.

2. The antibody of claim 1, wherein the HCDR1 comprises SEQ ID NO: 1, the HCDR2 comprises SEQ ID NO: 2, and the HCDR3 comprises SEQ ID NO: 48; and wherein the LCDR1 comprises SEQ ID NO: 49, the LCDR2 comprises SEQ ID NO: 5, and LCDR3 comprises SEQ ID NO: 6.

3. The antibody of claim 2, wherein the HCDR1 comprises SEQ ID NO: 1, HCDR2 comprises SEQ ID NO: 2, and HCDR3 comprises SEQ ID NO: 3; and LCDR1 comprises SEQ ID NO: 4, LCDR2 comprises SEQ ID NO: 5, and LCDR3 comprises SEQ ID NO: 6.

4. The antibody of claim 3, wherein the VH comprises SEQ ID NO: 7 and the VL comprises SEQ ID NO: 8.

5. The antibody of claim 1, wherein the antibody is a human IgG1 isotype.

6. The antibody of claim 1, wherein the antibody comprises a HC comprising SEQ ID NO: 11, and a LC comprising SEQ ID NO: 11.

7. The antibody of claim 1, wherein the antibody is a human IgG4 isotype.

8. The antibody of claim 1, wherein the antibody is an agonist of human LAG-3.

9. The antibody of claim 1, wherein the antibody agonizes human LAG-3 as determined by an assay measuring the inhibition of human or cynomolgus CD8 cell proliferation.

10. The antibody of claim 1, wherein the antibody binds human LAG-3 ECD of SEQ ID NO: 58 or SEQ ID NO: 59 with a dissociation equilibrium constant (K_{D}) of less than or equal to 100 nM.

11. The antibody of claim 1, wherein the antibody binds human LAG-3 ECD of SEQ ID NO: 58 or SEQ ID NO: 59 with a dissociation equilibrium constant (K_{D}) of less than or equal to 5 nM.

12. The antibody of claim 1, wherein the antibody binds to human LAG-3 ECD of SEQ ID NO: 58 or SEQ ID NO: 59 with a K_{D} of about 1×10⁻⁷ M to about 1×10⁻¹¹ M.

13. The antibody of claim 1, wherein the antibody binds to human LAG-3 ECD of SEQ ID NO: 58 or SEQ ID NO: 59 with a K_{D} of between about 1×10⁻⁸ M and about 1×10⁻¹¹ M.

14. The antibody of claim 1, wherein the antibody binds to human LAG-3 ECD of SEQ ID NO: 58 or SEQ ID NO: 59 with a K_{D} of between about 1×10⁻⁹ M and about 1×10⁻¹¹ M.

15. The antibody of claim 1, wherein the antibody binds to human LAG-3 ECD of SEQ ID NO: 58 or SEQ ID NO: 59 with a K_{D} of between about 1×10⁻¹⁰ M and about 1×10⁻¹¹ M.

16. The antibody of claim 1, wherein the K_{D} is determined by Surface Plasmon Resonance (SPR) at 37° C. assay.

17. A nucleic acid comprising a DNA sequence encoding one or both of SEQ ID NO: 11 and SEQ ID NO: 12.

18. A vector comprising the nucleic acid of claim 17.

19. A composition comprising a first vector comprising a nucleic acid sequence encoding SEQ ID NO: 11 and a second vector comprising a nucleic acid sequence encoding SEQ ID NO: 12.

20. A cell comprising the vector of claim 18.

21. A cell comprising the composition of claim 19.

22. The cell of claim 20, wherein the cell is a mammalian cell.

23. A process of producing an antibody, comprising culturing the cell of claim 20 under conditions such that the antibody is expressed, and recovering the expressed antibody from the culture medium.

24. An antibody produced by culturing the cell of claim 20 under conditions such that the antibody is expressed, and recovering the expressed antibody from the culture medium.

25. A composition comprising the antibody of claim 1.

26. A method of treating autoimmune disease in a subject in need thereof, comprising administering to the subject a therapeutically effective amount of the anti-human LAG-3 agonist antibody of claim 8.

27. The method of claim 26, wherein the autoimmune disease is rheumatoid arthritis, psoriasis, ulcerative colitis, Type I diabetes mellitus, lupus nephritis or systemic lupus erythematosus.

28. A pharmaceutical composition comprising the anti-human LAG-3 agonist antibody of claim 8, for use in treating rheumatoid arthritis, psoriasis, ulcerative colitis, Type I diabetes mellitus, lupus nephritis or systemic lupus erythematosus.


SEQUENCE LISTING (75 sequences)

SEQ ID NO: 1 - protein, 15 aa, Synthetic Construct
TVSGGSISSGGYYWS

SEQ ID NO: 2 - protein, 16 aa, Synthetic Construct
YIYYSGITYYNPSLKS

SEQ ID NO: 3 - protein, 11 aa, Synthetic Construct
ARSAVTTPFDY

SEQ ID NO: 4 - protein, 17 aa, Synthetic Construct
KSSQSILYSSDNKNYLA

SEQ ID NO: 5 - protein, 8 aa, Synthetic Construct
YWASTRES

SEQ ID NO: 6 - protein, 9 aa, Synthetic Construct
QQYYSPPWT

SEQ ID NO: 7 - protein, 119 aa, Synthetic Construct
QVQLQESGPGLVKPSQTLSLTCTVSGGSISSGGYYWSWIRQHPGKGLEWIGYIYYSGITYYNPSLKSRVTISVDTSKNQFSLKLSSVTAADTAVYYCARSAVTTPFDYWGQGTLVTVSS

SEQ ID NO: 8 - protein, 113 aa, Synthetic Construct
DIVMTQSPDSLAVSLGERATINCKSSQSILYSSDNKNYLAWYQQKPGQPPKLLIYWASTRESGVPDRFSGSGSGTDFTLTISSLQAEDVAVYYCQQYYSPPWTFGQGTKVEIK

SEQ ID NO: 9 - protein, 330 aa, Synthetic Construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 107 aa, Synthetic Construct [sequence omitted]

SEQ ID NO: 11 - protein, 449 aa, Synthetic Construct
QVQLQESGPGLVKPSQTLSLTCTVSGGSISSGGYYWSWIRQHPGKGLEWIGYIYYSGITYYNPSLKSRVTISVDTSKNQFSLKLSSVTAADTAVYYCARSAVTTPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 220 aa, Synthetic Construct
DIVMTQSPDSLAVSLGERATINCKSSQSILYSSDNKNYLAWYQQKPGQPPKLLIYWASTRESGVPDRFSGSGSGTDFTLTISSLQAEDVAVYYCQQYYSPPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - DNA, 1347 bp, Synthetic Construct
  source 1..1347: mol_type = other DNA
caggtgcagctgcaggagtcgggcccaggactggtgaagccttcacagaccctgtccctcacctgcactgtctctggtggctccatcagcagtggtggttactactggagctggatccgccagcacccagggaagggcctggagtggattgggtacatctattacagtgggataacctactacaacccgtccctcaagagtcgagttaccatatcagtagacacgtctaagaaccagttctccctgaagctgagctctgtgactgccgcggacacggccgtgtattactgtgcgagatcagcagtgaccacccccttcgactactggggacagggaactctggtgacggtgtcctccgccagcaccaagggcccatcggtcttccccctggcaccctcctccaagagcacctctgggggcacagcggccctgggctgcctggtcaaggactacttccccgaaccggtgacggtgtcgtggaactcaggcgcactgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtatgtggacggcgtggaggtgcataatgccaagacaaagccgcgggaggagcagtacaacagcacgtaccgtgtggtcagcgtcctcaccgtcctgcaccaagactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaagtcagcctgacctgcctggtcaaaggcttctatcccagcgacatcgccgtggagtgggagagcaatgggcagccggagaacaactacaagaccacgcctcccgtgctggactccgacggctccttcttcctctattccaagctcaccgtggacaagagcaggtggcagcaggggaacgtcttctcatgctccgtgatgcatgaggctctgcacaaccactacacgcagaagagcctctccctgtctccgggcaaa

SEQ ID NO: 14 - DNA, 660 bp, Synthetic Construct
  source 1..660: mol_type = other DNA
gacatcgtgatgacccagtctccagactccctggctgtgtctctgggcgagagggccaccatcaactgcaagtccagccagagtatattatacagctccgacaataagaactacttagcttggtaccagcagaaaccaggacagcctcctaagctgctcatttactgggcatctacccgggaatccggggtccctgaccgattcagtggcagcgggtctgggacagatttcactctcaccatcagcagcctgcaggctgaagatgtggcagtttattactgtcagcagtactactcaccaccatggacgttcgggcagggaactaaggtggaaataaagcgaaccgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgc

SEQ ID NO: 15 - protein, 11 aa, Synthetic Construct
ASSTVTTPFDY

SEQ ID NO: 16 - protein, 17 aa, Synthetic Construct
KSSQSVLYSSNNKNYLA

SEQ ID NO: 17 - protein, 119 aa, Synthetic Construct
  VARIANT 1: note = X at position 1 is Q, pyroglutamate, or absent
XVQLQESGPGLVKPSQTLSLTCTVSGGSISSGGYYWSWIRQHPGKGLEWIGYIYYSGITYYNPSLKSRVTISVDTSKNQFSLKLSSVTAADTAVYYCASSTVTTPFDYWGQGTLVTVSS

SEQ ID NO: 18 - protein, 113 aa, Synthetic Construct
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQYYSPPWTFGQGTKVEIK

SEQ ID NO: 19 - protein, 449 aa, Synthetic Construct
  VARIANT 1: note = X at position 1 is Q, pyroglutamate, or absent
  VARIANT 449: note = X at position 449 is K or absent
XVQLQESGPGLVKPSQTLSLTCTVSGGSISSGGYYWSWIRQHPGKGLEWIGYIYYSGITYYNPSLKSRVTISVDTSKNQFSLKLSSVTAADTAVYYCASSTVTTPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGX

SEQ ID NO: 20 - protein, 220 aa, Synthetic Construct
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQYYSPPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 13 aa, Synthetic Construct
TVSGVSITSYYWS

SEQ ID NO: 22 - protein, 16 aa, Synthetic Construct
RIFTSGSTNYNPSLKS

SEQ ID NO: 23 - protein, 7 aa, Synthetic Construct
ARYDGME

SEQ ID NO: 24 - protein, 12 aa, Synthetic Construct
RASQSVSSSYLA

SEQ ID NO: 25 - protein, 8 aa, Synthetic Construct
YGASSRAT

SEQ ID NO: 26 - protein, 9 aa, Synthetic Construct
QQYGSSPLT

SEQ ID NO: 27 - protein, 113 aa, Synthetic Construct
  VARIANT 1: note = X at position 1 is Q, pyroglutamate, or absent
XVQLQESGPGLVKPSETLSLTCTVSGVSITSYYWSWIRQPAGKGLEWIGRIFTSGSTNYNPSLKSRVTMSVDTSKNQFSLKLSSVTAADTAVYYCARYDGMEWGQGTLVTVSS

SEQ ID NO: 28 - protein, 108 aa, Synthetic Construct
  VARIANT 1: note = X at position 1 is E, pyroglutamate, or absent
XIVLTQSPGTLSLSPGERATLSCRASQSVSSSYLAWYQQKPGQAPRLLIYGASSRATGIPDRFSGSGSGTDFTLTISRLEPEDFAVYYCQQYGSSPLTFGQGTKVEIK

SEQ ID NO: 29 - protein, 443 aa, Synthetic Construct
  VARIANT 1: note = X at position 1 is Q, pyroglutamate, or absent
  VARIANT 443: note = X at position 443 is K or absent
XVQLQESGPGLVKPSETLSLTCTVSGVSITSYYWSWIRQPAGKGLEWIGRIFTSGSTNYNPSLKSRVTMSVDTSKNQFSLKLSSVTAADTAVYYCARYDGMEWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGX

SEQ ID NO: 30 - protein, 215 aa, Synthetic Construct
  VARIANT 1: note = X at position 1 is E, pyroglutamate, or absent
XIVLTQSPGTLSLSPGERATLSCRASQSVSSSYLAWYQQKPGQAPRLLIYGASSRATGIPDRFSGSGSGTDFTLTISRLEPEDFAVYYCQQYGSSPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 31 - protein, 13 aa, Synthetic Construct
TVSGGSIRSYYWS

SEQ ID NO: 32 - protein, 8 aa, Synthetic Construct
ARYEGFDV

SEQ ID NO: 33 - protein, 9 aa, Synthetic Construct
QQYGSSPIT

SEQ ID NO: 34 - protein, 114 aa, Synthetic Construct
  VARIANT 1: note = X at position 1 is Q, pyroglutamate, or absent
XVQLQESGPGLVKPSETLSLTCTVSGGSIRSYYWSWIRQPAGKGLEWIGRIFTSGSTNYNPSLKSRVTMSVDTSKNQFSLKLSSVTAADTAVYYCARYEGFDVWGQGTLVTVSS

SEQ ID NO: 35 - protein, 108 aa, Synthetic Construct
  VARIANT 1: note = X at position 1 is E, pyroglutamate, or absent
XIVLTQSPGTLSLSPGERATLSCRASQSVSSSYLAWYQQKPGQAPRLLIYGASSRATGIPDRFSGSGSGTDFTLTISRLEPEDFAVYYCQQYGSSPITFGQGTKVEIK

SEQ ID NO: 36 - protein, 444 aa, Synthetic Construct
  VARIANT 1: note = X at position 1 is Q, pyroglutamate, or absent
  VARIANT 444: note = X at position 444 is K or absent
XVQLQESGPGLVKPSETLSLTCTVSGGSIRSYYWSWIRQPAGKGLEWIGRIFTSGSTNYNPSLKSRVTMSVDTSKNQFSLKLSSVTAADTAVYYCARYEGFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGX

SEQ ID NO: 37 - protein, 215 aa, Synthetic Construct
  VARIANT 1: note = X at position 1 is E, pyroglutamate, or absent
XIVLTQSPGTLSLSPGERATLSCRASQSVSSSYLAWYQQKPGQAPRLLIYGASSRATGIPDRFSGSGSGTDFTLTISRLEPEDFAVYYCQQYGSSPITFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 38 - protein, 13 aa, Synthetic Construct
AASGFTFSSFAMS

SEQ ID NO: 39 - protein, 17 aa, Synthetic Construct
AISASGGRTYYADSVKG

SEQ ID NO: 40 - protein, 15 aa, Synthetic Construct
AKDEPYSGSYHGLDV

SEQ ID NO: 41 - protein, 16 aa, Synthetic Construct
RSSQSLLHSDGYNYLD

SEQ ID NO: 42 - protein, 8 aa, Synthetic Construct
YLGSNRAS

SEQ ID NO: 43 - protein, 9 aa, Synthetic Construct
MQAQQTPLT

SEQ ID NO: 44 - protein, 122 aa, Synthetic Construct
  VARIANT 1: note = X at position 1 is Q, pyroglutamate, or absent
XVQLVESGGGLVQPGGSLRLSCAASGFTFSSFAMSWVRQAPGKGLEWVSAISASGGRTYYADSVKGRFTISRDNSKNTLYLQMNSLRAEDTAVYYCAKDEPYSGSYHGLDVWGQGTLVTVSS

SEQ ID NO: 45 - protein, 112 aa, Synthetic Construct
DIVMTQSPLSLPVTPGEPASISCRSSQSLLHSDGYNYLDWYLQKPGQSPQLLIYLGSNRASGVPDRFSGSGSGTDFTLKISRVEAEDVGVYYCMQAQQTPLTFGQGTKVEIK

SEQ ID NO: 46 - protein, 452 aa, Synthetic Construct
  VARIANT 1: note = X at position 1 is Q, pyroglutamate, or absent
  VARIANT 452: note = X at position 452 is K or absent
XVQLVESGGGLVQPGGSLRLSCAASGFTFSSFAMSWVRQAPGKGLEWVSAISASGGRTYYADSVKGRFTISRDNSKNTLYLQMNSLRAEDTAVYYCAKDEPYSGSYHGLDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGX

SEQ ID NO: 47 - protein, 219 aa, Synthetic Construct
DIVMTQSPLSLPVTPGEPASISCRSSQSLLHSDGYNYLDWYLQKPGQSPQLLIYLGSNRASGVPDRFSGSGSGTDFTLKISRVEAEDVGVYYCMQAQQTPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 48 - protein, 11 aa, Synthetic Construct
  VARIANT 2: note = X at position 2 is R or S
  VARIANT 4: note = X at position 4 is A or T
AXSXVTTPFDY

SEQ ID NO: 49 - protein, 17 aa, Synthetic Construct
  VARIANT 6: note = X at position 6 is I, or V
  VARIANT 11: note = X at position 11 is D, or N
KSSQSXLYSSXNKNYLA

SEQ ID NO: 50 - protein, 119 aa, Synthetic Construct
  VARIANT 99: note = X at position 99 is R, or S
  VARIANT 101: note = X at position 101 is A, or T
  VARIANT 1: note = X at position 1 is Q, pyroglutamate, or absent
XVQLQESGPGLVKPSQTLSLTCTVSGGSISSGGYYWSWIRQHPGKGLEWIGYIYYSGITYYNPSLKSRVTISVDTSKNQFSLKLSSVTAADTAVYYCAXSXVTTPFDYWGQGTLVTVSS

SEQ ID NO: 51 - protein, 113 aa, Synthetic Construct
  VARIANT 29: note = X at position 29 is I, or V
  VARIANT 34: note = X at position 34 is D, or N
DIVMTQSPDSLAVSLGERATINCKSSQSXLYSSXNKNYLAWYQQKPGQPPKLLIYWASTRESGVPDRFSGSGSGTDFTLTISSLQAEDVAVYYCQQYYSPPWTFGQGTKVEIK

SEQ ID NO: 52 - protein, 13 aa, Synthetic Construct
  VARIANT 5: note = X at position 5 is G, or V
  VARIANT 8: note = X at position 8 is R, or T
TVSGXSIXSYYWS

SEQ ID NO: 53 - protein, 8 aa, Synthetic Construct
  VARIANT 4: note = X at position 4 is E, or D
  VARIANT 6: note = X at position 6 is F, or M
  VARIANT 7: note = X at position 7 is D, or E
  VARIANT 8: note = X at position 8 is V, or absent
ARYXGXXX

SEQ ID NO: 54 - protein, 9 aa, Synthetic Construct
  VARIANT 8: note = X at position 8 is I, or L
QQYGSSPXT

SEQ ID NO: 55 - protein, 114 aa, Synthetic Construct
  VARIANT 27: note = X at position 27 is G, or V
  VARIANT 30: note = X at position 30 is R, or T
  VARIANT 99: note = X at position 99 is E, or D
  VARIANT 101: note = X at position 101 is F, or M
  VARIANT 102: note = X at position 102 is D, or E
  VARIANT 103: note = X at position 103 is V, or absent
  VARIANT 1: note = X at position 1 is Q, pyroglutamate, or absent
XVQLQESGPGLVKPSETLSLTCTVSGXSIXSYYWSWIRQPAGKGLEWIGRIFTSGSTNYNPSLKSRVTMSVDTSKNQFSLKLSSVTAADTAVYYCARYXGXXXWGQGTLVTVSS

SEQ ID NO: 56 - protein, 108 aa, Synthetic Construct
  VARIANT 97: note = X at position 97 is I, or L
  VARIANT 1: note = X at position 1 is E, pyroglutamate, or absent
XIVLTQSPGTLSLSPGERATLSCRASQSVSSSYLAWYQQKPGQAPRLLIYGASSRATGIPDRFSGSGSGTDFTLTISRLEPEDFAVYYCQQYGSSPXTFGQGTKVEIK

SEQ ID NO: 57 - protein, 525 aa, Homo Sapiens
MWEAQFLGLLFLQPLWVAPVKPLQPGAEVPVVWAQEGAPAQLPCSPTIPLQDLSLLRRAGVTWQHQPDSGPPAAAPGHPLAPGPHPAAPSSWGPRPRRYTVLSVGPGGLRSGRLPLQPRVQLDERGRQRGDFSLWLRPARRADAGEYRAAVHLRDRALSCRLRLRLGQASMTASPPGSLRASDWVILNCSFSRPDRPASVHWFRNRGQGRVPVRESPHHHLAESFLFLPQVSPMDSGPWGCILTYRDGFNVSIMYNLTVLGLEPPTPLTVYAGAGSRVGLPCRLPAGVGTRSFLTAKWTPPGGGPDLLVTGDNGDFTLRLEDVSQAQAGTYTCHIHLQEQQLNATVTLAIITVTPKSFGSPGSLGKLLCEVTPVSGQERFVWSSLDTPSQRSFSGPWLEAQEAQLLSQPWQCQLYQGERLLGAAVYFTELSSPGAQRSGRAPGALPAGHLLLFLILGVLSLLLLVTGAFGFHLWRRQWRPRRFSALEQGIHPPQAQSKIEELEQEPEPEPEPEPEPEPEPEPEQL

SEQ ID NO: 58 - protein, 438 aa, Homo Sapiens
LQPGAEVPVVWAQEGAPAQLPCSPTIPLQDLSLLRRAGVTWQHQPDSGPPAAAPGHPLAPGPHPAAPSSWGPRPRRYTVLSVGPGGLRSGRLPLQPRVQLDERGRQRGDFSLWLRPARRADAGEYRAAVHLRDRALSCRLRLRLGQASMTASPPGSLRASDWVILNCSFSRPDRPASVHWFRNRGQGRVPVRESPHHHLAESFLFLPQVSPMDSGPWGCILTYRDGFNVSIMYNLTVLGLEPPTPLTVYAGAGSRVGLPCRLPAGVGTRSFLTAKWTPPGGGPDLLVTGDNGDFTLRLEDVSQAQAGTYTCHIHLQEQQLNATVTLAIITVTPKSFGSPGSLGKLLCEVTPVSGQERFVWSSLDTPSQRSFSGPWLEAQEAQLLSQPWQCQLYQGERLLGAAVYFTELSSPGAQRSGRAPGALPAGHGGGGSHHHHHH

SEQ ID NO: 59 - protein, 438 aa, Synthetic Construct; identical to SEQ ID NO: 58

SEQ ID NO: 60 - protein, 439 aa, Synthetic Construct
PQPGAEISVVWAQEGAPAQLPCSPTIPLQDLSLLRRAGVTWQHQPDSGPPAPAPGHPPVPGHRPAAPYSWGPRPRRYTVLSVGPGGLRSGRLPLQPRVQLDERGRQRGDFSLWLRPARRADAGEYRATVHLRDRALSCRLRLRVGQASMTASPPGSLRTSDWVILNCSFSRPDRPASVHWFRSRGQGRVPVQGSPHHHLAESFLFLPHVGPMDSGLWGCILTYRDGFNVSIMYNLTVLGLEPATPLTVYAGAGSRVELPCRLPPAVGTQSFLTAKWAPPGGGPDLLVAGDNGDFTLRLEDVSQAQAGTYICHIRLQGQQLNATVTLAIITVTPKSFGSPGSLGKLLCEVTPASGQEHFVWSPLNTPSQRSFSGPWLEAQEAQLLSQPWQCQLHQGERLLGAAVYFTELSSPGAQRSGRAPGALRAGHAAAHHHHHHSGS

SEQ ID NO: 61 - protein, 94 aa, Synthetic Construct
QASMTASPPGSLRASDWVILNCSFSRPDRPASVHWFRNRGQGRVPVRESPHHHLAESFLFLPQVSPMDSGPWGCILTYRDGFNVSIMYNLTVLG

SEQ ID NO: 62 - protein, 32 aa, Synthetic Construct
PDRPASVHWFRNRGQGRVPVRESPHHHLAESF

SEQ ID NO: 63 - protein, 7 aa, Synthetic Construct
PQVSPMD

SEQ ID NO: 64 - protein, 10 aa, Synthetic Construct
SPHHHLAESF

SEQ ID NO: 65 - protein, 15 aa, synthetic construct
  VARIANT 13: note = X at position 13 is F or Y
TVSGGSISSGGYXWS

SEQ ID NO: 66 - protein, 17 aa, synthetic construct
  VARIANT 6: note = X at position 6 is I or V
  VARIANT 11: note = X at position 11 is is D or N
  VARIANT 13: note = X at position 13 is R or K
KSSQSXLYSSXNXNYLA

SEQ ID NO: 67 - protein, 8 aa, synthetic construct
  VARIANT 7: note = X at position 7 is D or E
YWASTRXS

SEQ ID NO: 68 - protein, 9 aa, synthetic construct
  VARIANT 1: note = X at position 1 is H or Q
XQYYSPPWT

SEQ ID NO: 69 - protein, 119 aa, synthetic construct
  VARIANT 1: note = X at position 1 is Q, pyroglutamate, or absent
  VARIANT 35: note = X at position 35 is F or Y
  VARIANT 69: note = X at position 69 is L or V
  VARIANT 83: note = X at position 83 is N or K
  VARIANT 99: note = X at position 99 is R or S
  VARIANT 101: note = X at position 101 is A or T
XVQLQESGPGLVKPSQTLSLTCTVSGGSISSGGYXWSWIRQHPGKGLEWIGYIYYSGITYYNPSLKSRXTISVDTSKNQFSLXLSSVTAADTAVYYCAXSXVTTPFDYWGQGTLVTVSS

SEQ ID NO: 70 - protein, 113 aa, synthetic construct
  VARIANT 9: note = X at position 9 is A or S
  VARIANT 36: note = X at position 36 is R or K
  VARIANT 42: note = X at position 42 is F or Y
  VARIANT 44: note = X at position 44 is L or Q
  VARIANT 61: note = X at position 61 is D or E
  VARIANT 80: note = X at position 80 is A or T
  VARIANT 95: note = X at position 95 is H or Q
DIVMTQSPDXLAVSLGERATINCKSSQSILYSSDNXNYLAWXQXKPGQPPKLLIYWASTRXSGVPDRFSGSGSGTDFTLXISSLQAEDVAVYYCXQYYSPPWTFGQGTKVEIK

SEQ ID NO: 71 - protein, 449 aa, synthetic construct
  VARIANT 1: note = X at position 1 is Q, pyroglutamate, or absent
  VARIANT 35: note = X at position 35 is F or Y
  VARIANT 69: note = X at position 69 is L or V
  VARIANT 83: note = X at position 83 is N or K
  VARIANT 236: note = X at positon 236 is L or A
  VARIANT 237: note = X at position 237 is L or A
  VARIANT 267: note = X at position 267 is D or S
  VARIANT 449: note = X at position 449 is K or absent
XVQLQESGPGLVKPSQTLSLTCTVSGGSISSGGYXWSWIRQHPGKGLEWIGYIYYSGITYYNPSLKSRXTISVDTSKNQFSLXLSSVTAADTAVYYCARSAVTTPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEXXGGPSVFLFPPKPKDTLMISRTPEVTCVVVXVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGX

SEQ ID NO: 72 - protein, 220 aa, synthetic construct
  VARIANT 10: note = X at position 10 is A or S
  VARIANT 29: note = X at position 29 is I or V
  VARIANT 36: note = X at position 36 is R or K
  VARIANT 42: note = X at position 42 is F or Y
  VARIANT 44: note = X at position 44 is L or Q
  VARIANT 61: note = X at position 61 is D or E
  VARIANT 80: note = X at position 80 is A or T
  VARIANT 95: note = X at position 95 is H or Q
DIVMTQSPDXLAVSLGERATINCKSSQSXLYSSDNXNYLAWXQXKPGQPPKLLIYWASTRXSGVPDRFSGSGSGTDFTLXISSLQAEDVAVYYCXQYYSPPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 73 - protein, 119 aa, synthetic construct
  VARIANT 1: note = X at position 1 is Q, pyroglutamate, or absent
XVQLQESGPGLVKPSQTLSLTCTVSGGSISSGGYYWSWIRQHPGKGLEWIGYIYYSGITYYNPSLKSRVTISVDTSKNQFSLKLSSVTAADTAVYYCARSAVTTPFDYWGQGTLVTVSS

SEQ ID NO: 74 - protein, 330 aa, synthetic construct
  VARIANT 330: note = X at position 330 is K or absent
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGX

SEQ ID NO: 75 - protein, 449 aa, unidentified
  VARIANT 1: note = X at position 1 is Q, pyroglutamate, or absent
  VARIANT 449: note = X at position 449 is K or absent
XVQLQESGPGLVKPSQTLSLTCTVSGGSISSGGYYWSWIRQHPGKGLEWIGYIYYSGITYYNPSLKSRVTISVDTSKNQFSLKLSSVTAADTAVYYCARSAVTTPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGX